(19) United States
(12) Patent Application Publication
(10) Pub. No.: US 2023/0404887 A1
(43) Pub. Date: Dec. 21, 2023

(54) COMPOSITIONS TO LIMIT OR ELIMINATE THE PERCEPTION OF SWEAT MALODOR

(71) Applicant: Firmenich SA, Satigny (CH)

(72) Inventors: Christine VUILLEUMIER, Satigny (CH); Gary MARR, Plainsboro, NJ (US); Jessica BRANN, Plainsboro, NJ (US); Lily WU, Plainsboro, NJ (US); Zhenan WU, Plainsboro, NJ (US); Nicholas O'LEARY, Plainsboro, NJ (US); Benjamin SMITH, Plainsboro, NJ (US)

(73) Assignee: Firmenich SA, Satigny (CH)

(21) Appl. No.: 18/247,402

(22) PCT Filed: Oct. 22, 2021
(86) PCT No.: PCT/EP2021/079432

§ 371 Date: Mar. 30, 2023

Related U.S. Application Data
(60) Provisional application No. 63/104,591, filed on Oct. 23, 2020.

(30) Foreign Application Priority Data
Nov. 17, 2020 (EP) 20208004.0

(51) Int. Cl. A61K 8/49; A61Q 15/00; A61K 8/04; A61K 8/35

(52) U.S. Cl. CPC A61K 8/4973; A61Q 15/00; A61K 8/046; A61K 8/35; A61K 8/4926; A61K 8/498; A61K 2800/87; A61K 2800/874

(57) ABSTRACT

The various aspects presented herein relate to the perfumery industry. More particularly, the various aspects presented herein relate to the use of volatile compositions to limit, decrease or eliminate the perception of sweat malodor. Such compositions include at least one malodor antagonist optionally combined with perfuming ingredients. Such compositions, their use in combination with delivery systems and their applications in consumer products are also objects of the present disclosure.

### FIELD OF THE INVENTION

The present invention relates to the use of volatile compositions to limit, decrease or eliminate the perception of sweat malodor. Such compositions and their applications in consumer products are also objects of the present invention.

### BACKGROUND

Malodorous smells exist in many environments and are experienced in our daily life. Malodors are frequently complex mixtures of more than one malodorant compound which may typically include various amines, thiols, sulfides, short chain aliphatic and olefinic acids, e.g. fatty acids, and derivatives thereof. For example, body related malodors typically include short chain fatty acids, such as 3-methyl-3-hydroxyhexanoic acid, 3-methylhexanoic acid, 3-2-methyl-2-hexenoic acid, 3-methyl-3-hydroxyhexanoic acid, 3-methyl-2-hexenoic acid, or 3-methyl-3-sulphanylhexanol (transpirol) (found in axilla malodors).

Malodors are not pleasant for humans and therefore there is a constant need for malodor counteracting (MOC) compositions and/or ingredients for decreasing or suppressing the perception of malodors.

The present invention provides methods to counteract sweat malodor using volatile compositions that limit, decrease or eliminate the perception of malodor. Such compositions, their use in combination with delivery systems and their applications in consumer products are also objects of the present invention.

### SUMMARY OF THE INVENTION

One aspect presented herein provides a method to inhibit, reduce, or suppress the perception of a sweat malodor in a subject, comprising contacting the subject with a composition comprising at least one compound selected from the group consisting of:

(+−)-2,6-dimethyl-2-octanol; allyl heptanoate; (2E)-3-phenyl-2-propen-1-ol; (+−)-2,2,5-trimethyl-5-pentylcyclopentanone; (+−)-3,7-dimethyl-3-octanol; methyl 2-octynoate; (+−)-2-methylundecanal; (+−)-methyl 2,2-dimethyl-6-methylidenecyclohexanecarboxylate; ethyl (E)-3-phenyl-2-propenoate; methyl (E)-3-phenyl-2-propenoate; 1−(2-naphthyl)ethenone; (10E)-oxacycloheptadec-10-en-2-one; 4-(4-hydroxyphenyl)-2-butanone; (+−)-6-methoxy-2,6-dimethylheptanal; ethyl tricyclo[5.2.1.0. (2,6)]decane-2-carboxylate; 4-methoxybenzyl acetate; (3Z)-3-hexen-1-ol; pentyl 2-hydroxybenzoate; allyl hexanoate; tricyclo[5.2.1.0(2,6)]dec-3/4-en-8-yl propanoate; (2RS,4SR)-4-methyl-2-phenyltetrahydro-2H-pyran; (+−)-3-(4-methoxyphenyl)-2-methylprop anal; (+−)-3-(1,3-benzodioxol-5-yl)-2-methylprop anal; 2,6-dimethyl-4-heptanol; benzophenone; (2Z)-1,1-dimethoxy-3,7-dimethyl-2,6-octadiene; 4-isopropylbenzaldehyde; (+−)-4-nonanolide; (E)-4-decenal; (Z)-3,7-dimethyl-1,3,6-octatriene; 3-phenylprop anal; 2-methoxynaphthalene; 2-ethyl-3-hydroxy-4(4H)-pyranone; (+−)-2-(4-methyl-3-cyclohexen-1-yl)-2-propanyl acetate; (E)-3,7-dimethyl-2,6-octadien-1-ol; ethyl 2-methyl-1,3-dioxolane-2-acetate; 3-(2/4-ethylphenyl)-2,2-dimethylprop anal; (2RS,4 S R)-4-methyl-2-(2-methyl-1-propen-1-yl)tetrahydro-2H-pyran; (3 aRS,5 aSR,9 aSR,9bRS)-3a,6,6,9a-tetramethyldodecahydronaphtho[2,1-b] furan; (2,2-dimethoxyethyl)benzene; (+−)-(5E)-3-methyl-5-cyclopentadecen-1-one; 4-methoxybenzaldehyde; 10-undecenal; (2E)-2-nonenal; 4-phenyl-2-butanone; (+−)-(6E)-3,7-dimethyl-1,6-nonadien-3-yl acetate; ethyl 1RS,2 S R,3 SR,4SR-3-isopropylbicyclo[2.2.1] hept-ene-2-carboxylate; 3-ethoxy-4-hydroxybenzaldehyde; methyl 2-((1 RS,2RS)-3-oxo-2-pentylcyclopentyl)acetate; (+−)-2,6-dimethyl-7-octen-2-ol; (+−)-6-sec-butylquinoline; (+−)-3,7-dimethyl-6-octenal; methyl 2,4-dihydroxy-3,6-dimethylbenzoate; 4-cyclohexyl-2-methyl-2-butanol; (3E)-4-(2,6,6-trimethyl-1-cyclohexen-1-yl)-3-buten-2-one; (−)-(2E)-2-ethyl-4-[(1R)-2,2,3-trimethyl-3-cyclopenten-1-yl]-2-buten-1-ol; 1-(3,3/5,5-dimethyl-1-cyclohexen-1-yl)-4-penten-1-one; (+)-(3R,5Z)-3-methyl-5-cyclopentadecen-1-one; patchouli oil; thymol; (+−)-(3E)-4-(2,6,6-trimethyl-1/2-cyclohexen-1-yl)-3-buten-2-one; 2-(3-phenylpropyl)pyridine; (+−)-(2E)-1-(2,6,6-trimethyl-2-cyclohexen-1-yl)-2-buten-1-one; (+−)-3,5,6,6-tetramethyl-4-methylidene-2-heptanone; (4E)-3,4,5,6,6-pentamethyl-4-hepten-2-one; 3,4,5,6,6-pentamethyl-3-hepten-2-one; (2E,6Z)-2,6-nonadienal; (+−)-(E)-4-methyl-3-dec en-5-ol; 2-chromenone; (+−)-(3E)-3-methyl-4-(2,6,6-trimethyl-2-cyclohexen-1-yl)-3-buten-2-one; (+−)-(1E)-1-(2,6,6-trimethyl-2-cyclohexen-1-yl)-1-penten-3-one; (+−)-(2,5-dimethyl-2,3-dihydro-1H-inden-2-yl)methanol; (E)-2-ethoxy-5-(1-propenyl)phenol; undecanal; 1-[(1RS,2RS)-1,2,8,8-tetramethyl-1,2,3,4,5,6,7,8-octahydro-2-naphthalenyl] ethenone; 1-((2RS,3RS)-2,3,8,8-tetramethyl-1,2,3,4,5,6,7,8-octahydronaphthalen-2-yl)ethenone; 1-[(2RS,3RS,8aRS)-2,3,8,8-tetramethyl-1,2,3,5,6,7,8,8a-octahydro-2-naphthalenyl]ethenone; 1-[(1RS,2RS,8aSR)-1,2,8,8-tetramethyl-1,2,3,5,6,7,8,8a-octahydro-2-naphthalenyl]ethenone; 1-[(2RS,3RS,8aRS)-2,3,8,8-tetramethyl-1,2,3,4,6,7,8,8a-octahydro-2-naphthalenyl]ethenone; 2-phenylethanol, (4Z)-4-dodecenal; octanal; decanal; (+−)-tetrahydro-2-isobutyl-4-methyl-4 (2H)-pyranol; (1RS,2RS)-2,4-dimethyl-3-cyclohexene-1-carbaldehyde, (1RS,2SR)-2,4-dimethyl-3-cyclohexene-1-carbaldehyde; and combinations thereof;

wherein the perception of the sweat malodor is inhibited, reduced, or suppressed.

In one aspect, the present invention encompasses contacting a subject with a composition comprising of from about 1% to about 100% of (+−)-2,2,5-trimethyl-5-pentylcyclopentanone; wherein the subject's perception of sweat malodor is inhibited.

In a further aspect, the present invention encompasses contacting a subject with a composition comprising from about 2% to about 100% of a compound selected from the group consisting of: (2E)-3-phenyl-2-propen-1-ol; methyl (E)-3-phenyl-2-propenoate; 1-(2-naphthyl)ethenone; (10E)-oxacycloheptadec-10-en-2-one; 1-(2-naphthyl)ethenone; and combinations thereof;

wherein the subject's perception of sweat malodor is inhibited, reduced or suppressed.

In another aspect, the present invention encompasses contacting a subject with from about 3% to about 100% of a compound selected from the group consisting of: (+−)-2,6-dimethyl-2-octanol; 4-methoxybenzyl acetate; tricyclo[5.2.1.0(2,6)]dec-3/4-en-8-yl propanoate; and combinations thereof;

wherein the subject's perception of sweat malodor is inhibited, reduced or suppressed.

A method according to the present invention includes contacting a subject with from about 0.1% to about 100% of a compound selected from the group consisting of: (2RS,4SR)-4-methyl-2-phenyltetrahydro-2H-pyran; 3-(6,6-dimethyl-bicyclo[3.1.1]hept-2-en-2-yl)propanal; and combinations thereof;

wherein the subject's perception of sweat malodor is inhibited, reduced or suppressed.

The present invention includes contacting a subject with from about 7% to about 100% of a compound selected from the group consisting of: 4-(4-hydroxyphenyl)-2-butanone; allyl heptanoate; and combinations thereof;

wherein the subject's perception of sweat malodor is inhibited, reduced or suppressed.

In an aspect of the present invention, a method is provided for contacting a subject with from about 20% to about 100% of a compound selected from the group consisting of: pentyl 2-hydroxybenzoate; (+−)-3,7-dimethyl-3-octanol; and combinations thereof;

wherein the subject's perception of sweat malodor is inhibited, reduced or suppressed.

The present invention includes a method for contacting a subject with about 4% to about 100% of (+−)-3-(1,3-benzodioxol-5-yl)-2-methylpropanal; wherein the subject's perception of sweat malodor is inhibited, reduced or suppressed.

An aspect of the present invention encompasses a method for contacting a subject with about 0.06% to about 100% of methyl 2-octynoate; wherein the subject's perception of sweat malodor is inhibited, reduced or suppressed.

The present invention includes a method for contacting a subject with about 0.4% to about 100% of (+−)-methyl 2,2-dimethyl-6-methylidenecyclohexanecarboxylate; wherein the subject's perception of sweat malodor is inhibited, reduced or suppressed.

An aspect of the present invention includes a method for contacting a subject with about to about 100% of ethyl (E)-3-phenyl-2-propenoate; wherein the subject's perception of sweat malodor is inhibited, reduced or suppressed.

The present invention includes a method for contacting a subject with about 0.6% to about 100% of ethyl tricyclo[5.2.1.0.(2,6)]decane-2-carboxylate; wherein the subject's perception of sweat malodor is inhibited, reduced or suppressed.

A composition of the present invention may comprises a perfume ingredient.

A composition of the present invention may further comprise an excipient selected from the group consisting of: a humectant, an emollient an antioxidant, a pH adjustor, a coloring agent, a preservative, a thickening agent, a stabilizer, a propellant, a solvent, a cooling agent, and combinations thereof.

The present invention includes a consumer product comprising about 0.8 to about 5.0 wt % of the composition.

A consumer product of the present invention may be a deodorant or anti-perspirant. The deodorant or anti-perspirant may be a body deodorant spray, a roll-on deodorant, a deodorant stick, a deodorant cream, an antiperspirant spray, an antiperspirant stick, a roll-on antiperspirant liquid, an antiperspirant stick, or an antiperspirant cream.

Aspects of the present invention include use of the composition for inhibiting, reducing or suppressing perception of a sweat malodor in a subject.

### BRIEF DESCRIPTION OF THE FIGURES

FIG. 1 shows the headspace concentrations of malodor reducing materials resulting in malodor reduction when tested against 3 components present in axillary sweat malodor by an expert perfumer, along with the corresponding relative perceived intensities of the materials. Evaluations were performed using a dynamic olfactometer. The malodor components are shown in the legend, abbreviated as 3H3MHA (3-methyl-3-hydroxyhexanoic acid), 3M2HA (3-methyl-2-hexenoic acid), 3M3SH (3-methyl-3-sulphanylhexanol), and were held at a fixed intensity of 0.5 (mid-intensity) in the tests.

### DETAILED DESCRIPTION

Some aspects presented herein provide a method to inhibit, reduce, or suppress the perception of a sweat malodor in a subject, comprising contacting the subject with a composition comprising at least one compound in an amount sufficient to inhibit, reduce, suppress the subject's perception of the sweat malodor.

A “sweat malodor” refers to an odor that originates on a human body and includes sweat malodor that is transferred to another substrate such as a textile. This may include, for example, the odor of worn socks, or the odor of worn sportswear.

Compounds According to Some Aspects of the Present Disclosure

In some aspects, the at least one compound may be modified to prolong, modify, increase, or enhance the olfactive and/or malodor counteractant benefit provided by the at least one compound. One example of the modification may be to generate a precursor compound able to release, during or after application, the at least one compound upon a suitable external stimulus, such as an enzyme, light, temperature or a change of pH. A precursor compound able to release a perfuming ingredient under a suitable trigger is also known as properfume or profragrance. In some aspects, the profragrance does not provide any olfactive and/or malodor counteractant benefits.

In some aspects, the profragrance may be in a form of a β-thio carbonyl profragrance, when the at least one compound comprises at least one enone or enal functional group. Examples of methods used to generate said profragrance are described in International Patent Application Publication No. WO2003/049666. For example, 4-(2,6,6-trimethyl-1-cyclohexen-1-yl)-3-buten-2-one and 4-(2,6,6-trimethyl-2 such as -cyclohexen-1-yl)-3-buten-2-one may be in a form of a β-thio carbonyl profragrance such as 4-(dodecylthio)-4-(2,6,6-trimethylcyclohex-1/2-en-1-yl)butan-2-one (also known and referred to herein as Haloscent® I, trademark and origin: Firmenich SA).

Methods According to Some Aspects of the Present Disclosure

Some aspects presented herein provide a method to inhibit, reduce, or suppress the perception of a sweat malodor in a subject, comprising contacting the subject with a composition comprising at least one compound selected from the group consisting of: (+−)-2,6-dimethyl-2-octanol; allyl heptanoate; (2E)-3-phenyl-2-propen-1-ol; (+−)-2,2,5-trimethyl-5-pentylcyclopentanone; (+−)-3,7-dimethyl-3-octanol; methyl 2-octynoate; (+−)-2-methylundecanal; (+−)-methyl 2,2-dimethyl-6-methylidenecyclohexanecarboxylate; ethyl (E)-3-phenyl-2-propenoate; methyl (E)-3-phenyl-2-propenoate; 1-(2-naphthyl)ethenone; (10E)-oxacycloheptadec-10-en-2-one; 4-(4-hydroxyphenyl)-2-butanone; (+−)-6-methoxy-2,6-dimethylheptanal; ethyl tricyclo[5.2.1.0.(2,6)]decane-2-carboxylate; 4-methoxybenzyl acetate; (3Z)-3-hexen-1-ol; pentyl 2-hydroxybenzoate; allyl hexanoate; tricyclo[5.2.1.0(2,6)]dec-3-en-8-yl propanoate; (2RS,45R)-4-methyl-2-phenyltetrahydro-2H-pyran; (+−)-3-(4-methoxyphenyl)-2-methylprop anal; (+−)-3-(1,3-benzodioxol-5-yl)-2-methylprop anal; 2,6-dimethyl-4-heptanol; benzophenone; (2Z)-1,1-dimethoxy-3,7-dimethyl-2,6-octadiene; 4-isopropylbenzaldehyde; (+−)-4-nonanolide; (E)-4-decenal; (Z)-3,7-dimethyl-1,3,6-octatriene; 3-phenylpropanal; 2-methoxynaphthalene; 2-ethyl-3-hydroxy-4(4H)-pyranone; (+−)-2-(4-methyl-3-cyclohexen-1-yl)-2-propanyl acetate; (E)-3,7-dimethyl-2,6-octadien-1-ol; ethyl 2-methyl-1,3-dioxolane-2-acetate; 3-(4-ethylphenyl)-2,2-dimethylprop anal; (2R5,45R)-4-methyl-2-(2-methyl-1-propen-1-yl)tetrahydro-2H-pyran; (3aRS,5aSR,9aSR,9bRS)-3a,6,6,9a-tetramethyldodecahydronaphtho[2,1-b]furan; (2,2-dimethoxyethyl)benzene; (+−)-(5E)-3-methyl-5-cyclopentadecen-1-one; 4-methoxybenzaldehyde; 10-undecenal; (2E)-2-nonenal; 4-phenyl-2-butanone; (+−)-(6E)-3,7-dimethyl-1,6-nonadien-3-yl acetate; ethyl 1RS,2SR,3 SR,4SR-3-isopropylbicyclo[2.2.1]hept-5-ene-2-carboxylate; 3-ethoxy-4-hydroxybenzaldehyde; methyl 2-((1RS,2RS)-3-oxo-2-pentylcyclopentyl)acetate; (+−)-2,6-dimethyl-7-octen-2-ol; (+−)-6-sec-butylquinoline; (+−)-3,7-dimethyl-6-octenal; methyl 2,4-dihydroxy-3,6-dimethylbenzoate; 4-cyclohexyl-2-methyl-2-butanol; (3E)-4-(2,6,6-trimethyl-1-cyclohexen-1-yl)-3-buten-2-one; (−)-(2E)-2-ethyl-4-[(1R)-2,2,3-trimethyl-3-cyclopenten-1-yl]-2-buten-1-ol; 1-(3,3/5,5-dimethyl-1-cyclohexen-1-yl)-4-penten-1-one; (+)-(3R,5Z)-3-methyl-5-cyclopentadecen-1-one; patchouli oil; thymol; (+−)-(3E)-4-(2,6,6-trimethyl-1/2-cyclohexen-1-yl)-3-buten-2-one; 2-(3-phenylpropyl)pyridine; (+−)-(2E)-1-(2,6,6-trimethyl-2-cyclohexen-1-yl)-2-buten-1-one; (+−)-3,5,6,6-tetramethyl-4-methylidene-2-heptanone; (4E)-3,4,5,6,6-pentamethyl-4-hepten-2-one; 3,4,5,6,6-pentamethyl-3-hepten-2-one; (2E,6Z)-2,6-nonadienal; (+−)-(E)-4-methyl-3-dec en-5-ol; 2-chromenone; (+−)-(3E)-3-methyl-4-(2,6,6-trimethyl-2-cyclohexen-1-yl)-3-buten-2-one; (+−)-(1E)-1-(2,6,6-trimethyl-2-cyclohexen-1-yl)-1-penten-3-one; (+−)-(2,5-dimethyl-2,3-dihydro-1H-inden-2-yl)methanol; (E)-2-ethoxy-5-(1-propenyl)phenol; undecanal; 1-[(1RS,2RS)-1,2,8,8-tetramethyl-1,2,3,4,5,6,7,8-octahydro-2-naphthalenyl]ethenone; 1-((2RS,3RS)-2,3,8,8-tetramethyl-1,2,3,4,5,6,7,8-octahydronaphthalen-2-yl)ethenone; 1-[(2RS,3RS,8aRS)-2,3,8,8-tetramethyl-1,2,3,5,6,7,8,8a-octahydro-2-naphthalenyl]ethenone; 1-[(1RS,2RS,8aSR)-1,2,8,8-tetramethyl-1,2,3,5,6,7,8,8a-octahydro-2-naphthalenyl]ethenone; 1-[(2RS,3RS,8aRS)-2,3,8,8-tetramethyl-1,2,3,4,6,7,8,8a-octahydro-2-naphthalenyl]ethenone; 2-phenylethanol, (4Z)-4-dodecenal; octanal; decanal; (+−)-tetrahydro-2-isobutyl-4-methyl-4(2H)-pyranol; (1RS,2RS)-2,4-dimethyl-3-cyclohexene-1-carbaldehyde, (1RS,2SR)-2,4-dimethyl-3-cyclohexene-1-carbaldehyde; and combinations thereof;

wherein the perception of the sweat malodor is inhibited, reduced, or suppressed.

In some aspects, the subject is contacted by treating a surface with, or dispensing at least partly in the air, the at least one compound.

The expression “patchouli oil” as mentioned above, designates any oil extracted from patchouli, any terpenes fractions of patchouli oil or Clearwood® (origin: Firmenich SA, Geneva, Switzerland).

In some aspects, the composition may comprise at least two, three, four five or more of compound selected from the group consisting of:

(+−)-2,6-dimethyl-2-octanol; allyl heptanoate; (2E)-3-phenyl-2-propen-1-ol; (+−)-2,2,5-trimethyl-5-pentylcyclopentanone; (+−)-3,7-dimethyl-3-octanol; methyl 2-octynoate; (+−)-2-methylundecanal; (+−)-methyl 2,2-dimethyl-6-methylidenecyclohexanecarboxylate; ethyl (E)-3-phenyl-2-propenoate; methyl (E)-3-phenyl-2-propenoate; 1-(2-naphthyl)ethenone; (10E)-oxacycloheptadec-10-en-2-one; 4-(4-hydroxyphenyl)-2-butanone; (+−)-6-methoxy-2,6-dimethylheptanal; ethyl tricyclo[5.2.1.0.(2,6)]decane-2-carboxylate; 4-methoxybenzyl acetate; (3Z)-3-hexen-1-ol; pentyl 2-hydroxybenzoate; allyl hexanoate; tricyclo[5.2.1.0(2,6)]dec-3-en-8-yl propanoate; (2R5,45R)-4-methyl-2-phenyltetrahydro-2H-pyran; (+−)-3-(4-methoxyphenyl)-2-methylpropanal; (+−)-3-(1,3-benzodioxol-5-yl)-2-methylprop anal; 2,6-dimethyl-4-heptanol; benzophenone; (2Z)-1,1-dimethoxy-3,7-dimethyl-2,6-octadiene; 4-isopropylbenzaldehyde; (+−)-4-nonanolide; (E)-4-decenal; (Z)-3,7-dimethyl-1,3,6-octatriene; 3-phenylpropanal; 2-methoxynaphthalene; 2-ethyl-3-hydroxy-4(4H)-pyranone; (+−)-2-(4-methyl-3-cyclohexen-1-yl)-2-propanyl acetate; (E)-3,7-dimethyl-2,6-octadien-1-ol; ethyl 2-methyl-1,3-dioxolane-2-acetate; 3-(4-ethylphenyl)-2,2-dimethylprop anal; (2RS,4SR)-4-methyl-2-(2-methyl-1-propen-1-yl)tetrahydro-2H-pyran; (3aRS,5aSR,9aSR,9bRS)-3a,6,6,9a-tetramethyldodecahydronaphtho[2,1-b]furan; (2,2-dimethoxyethyl)benzene; (+−)-(5E)-3-methyl-5-cyclopentadecen-1-one; 4-methoxybenzaldehyde; 10-undecenal; (2E)-2-nonenal; 4-phenyl-2-butanone; (+−)-(6E)-3,7-dimethyl-1,6-nonadien-3-yl acetate; ethyl 1RS,2SR,3 SR,4SR-3-isopropylbicyclo[2.2.1]hept-5-ene-2-carboxylate; 3-ethoxy-4-hydroxybenzaldehyde; methyl 2-((1RS,2RS)-3-oxo-2-pentylcyclopentyl)acetate; (+−)-2,6-dimethyl-7-octen-2-ol; (+−)-6-sec-butylquinoline; (+−)-3,7-dimethyl-6-octenal; methyl 2,4-dihydroxy-3,6-dimethylbenzoate; 4-cyclohexyl-2-methyl-2-butanol; (3E)-4-(2,6,6-trimethyl-1-cyclohexen-1-yl)-3-buten-2-one; (−)-(2E)-2-ethyl-4-[(1R)-2,2,3-trimethyl-3-cyclopenten-1-yl]-2-buten-1-ol; 1-(3,3/5,5-dimethyl-1-cyclohexen-1-yl)-4-penten-1-one; (+)-(3R,5Z)-3-methyl-5-cyclopentadecen-1-one; patchouli oil; thymol; (+−)-(3E)-4-(2,6,6-trimethyl-1/2-cyclohexen-1-yl)-3-buten-2-one; 2-(3-phenylpropyl)pyridine; (+−)-(2E)-1-(2,6,6-trimethyl-2-cyclohexen-1-yl)-2-buten-1-one; (+−)-3,5,6,6-tetramethyl-4-methylidene-2-heptanone; (4E)-3,4,5,6,6-pentamethyl-4-hepten-2-one; 3,4,5,6,6-pentamethyl-3-hepten-2-one; (2E,6Z)-2,6-nonadienal; (+−)-(E)-4-methyl-3-dec en-5-ol; 2-chromenone; (+−)-(3E)-3-methyl-4-(2,6,6-trimethyl-2-cyclohexen-1-yl)-3-buten-2-one; (+−)-(1E)-1-(2,6,6-trimethyl-2-cyclohexen-1-yl)-1-penten-3-one; (+−)-(2,5-dimethyl-2,3-dihydro-1H-inden-2-yl)methanol; (E)-2-ethoxy-5-(1-propenyl)phenol; undecanal; 1-[(1RS,2RS)-1,2,8,8-tetramethyl-1,2,3,4,5,6,7,8-octahydro-2-naphthalenyl] ethenone; 1-((2RS,3RS)-2,3,8,8-tetramethyl-1,2,3,4,5,6,7,8-octahydronaphthalen-2-yl)ethenone; 1-[(2RS,3RS,8aRS)-2,3,8,8-tetramethyl-1,2,3,5,6,7,8,8a-octahydro-2-naphthalenyl]ethenone; 1-[(1RS,2RS,8aSR)-1,2,8,8-tetramethyl-1,2,3,5,6,7,8,8a-octahydro-2-naphthalenyl]ethenone; 1-[(2RS,3RS,8aRS)-2,3,8,8-tetramethyl-1,2,3,4,6,7,8,8a-octahydro-2-naphthalenyl]ethenone; 2-phenylethanol, (4Z)-4-dodecenal; octanal; decanal; (+−)-tetrahydro-2-isobutyl-4-methyl-4(2H)-pyranol; (1RS,2RS)-2,4-dimethyl-3-cyclohexene-1-carbaldehyde, (1RS,2SR)-2,4-dimethyl-3-cyclohexene-1-carbaldehyde; and combinations thereof;

According to any embodiments of the invention, the composition may comprise (3E)-4-(2,6,6-trimethyl-1-cyclohexen-1-yl)-3-buten-2-one in an amount of at least 4 wt. %, preferably of at least 6 wt. %, even more preferably of at least 10 wt. %, based on the total weight of the composition. Particularly, the composition may comprise (3E)-4-(2,6,6-trimethyl-1-cyclohexen-1-yl)-3-buten-2-one in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, even more preferably from 6 wt. % to 25 wt. % based on the total weight of the composition.

According to any embodiments of the invention, the composition may comprise (+−)-2-methylundecanal in an amount of at least 1.5 wt. %, preferably of at least 2 wt. %, even more preferably of at least 5 wt. %, based on the total weight of the composition. Particularly, the composition may comprise (+−)-2-methylundecanal in an amount of from 1.5 wt. % to 95 wt. %, preferably from 1.5 wt. % to 90 wt. %, preferably from 1.5 wt. % to 80 wt. %, preferably from 1.5 wt. % to 50 wt. %, preferably from 1.5 wt. % to 25 wt. %, preferably from 1.5 wt. % to wt. %, preferably from 1.5 wt. % to 5 wt. %, even more preferably from 2 wt. % to 5 wt. % based on the total weight of the composition.

According to any embodiments of the invention, the composition may comprise (−)-(2E)-2-ethyl-4-[(1R)-2,2,3-trimethyl-3-cyclopenten-1-yl]-2-buten-1-ol in an amount of at least 3 wt. %, preferably of at least 7 wt. %, even more preferably of at least 10 wt. %, based on the total weight of the composition. Particularly, the composition may comprise (−)-(2E)-2-ethyl-4-[(1R)-2,2,3-trimethyl-3-cyclopenten-1-yl]-2-buten-1-ol in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 10 wt. %, even more preferably from 7 wt. % to 10 wt. % based on the total weight of the composition.

According to any embodiments of the invention, the composition may comprise 143,3/5,5-dimethyl-1-cyclohexen-1-yl)-4-penten-1-one in an amount of at least 0.2 wt. %, preferably of at least 0.4 wt. %, even more preferably of at least 1 wt. %, based on the total weight of the composition. Particularly, the composition may comprise 1-(3,3/5,5-dimethyl-1-cyclohexen-1-yl)-4-penten-1-one in an amount of from 0.4 wt. % to 95 wt. %, preferably from 0.4 wt. % to wt. %, preferably from 0.4 wt. % to 80 wt. %, preferably from 0.4 wt. % to 50 wt. %, preferably from 0.4 wt. % to 25 wt. %, preferably from 0.4 wt. % to 10 wt. %, preferably from 0.4 wt. % to 8 wt. %, even more preferably from 2 wt. % to 8 wt. % based on the total weight of composition.

According to any embodiments of the invention, the composition may comprise methyl 2,4-dihydroxy-3,6-dimethylbenzoate in an amount of at least 1.5 wt. %, preferably of at least 2 wt. %, even more preferably of at least 5 wt. %, based on the total weight of the composition.

Particularly, the composition may comprise methyl 2,4-dihydroxy-3,6-dimethylbenzoate in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 10 wt. %, preferably from 2 wt. % to 5 wt. %, even more preferably from 2 wt. % to 4 wt. % based on the total weight of the composition.

According to any embodiments of the invention, the composition may comprise (+)-(3R,5Z)-3-methyl-5-cyclopentadecen-1-one in an amount of at least 0.1 wt. %, preferably of at least 0.6 wt. %, even more preferably of at least 1 wt. %, based on the total weight of the composition. Particularly, the composition may comprise (+)-(3R,5Z)-3-methyl-5-cyclopentadecen-1-one in an amount of from 0.1 wt. % to 95 wt. %, preferably from 0.1 wt. % to 90 wt. %, preferably from 0.1 wt. % to 80 wt. %, preferably from 0.1 wt. % to 50 wt. %, preferably from 0.1 wt. % to 25 wt. %, preferably from 0.1 wt. % to 10 wt. %, preferably from 0.1 wt. % to 8 wt. %, even more preferably from 0.6 wt. % to 8 wt. % based on the total weight of the composition.

According to any embodiments of the invention, the composition may comprise patchouli oil in an amount of at least 1 wt. %, preferably of at least 3 wt. %, even more preferably of at least 10 wt. %, based on the total weight of the composition. Particularly, the composition may comprise patchouli oil in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 20 wt. %, preferably from 5 wt. % to 20 wt. %, even more preferably from 8 wt. % to 15 wt. % based on the total weight of the composition.

According to any embodiments of the invention, the composition may comprise thymol in an amount of at least 0.1 wt. %, preferably of at least 0.5 wt. %, even more preferably of at least 2 wt. %, based on the total weight of the composition. Particularly, the composition may comprise thymol in an amount of from 1 wt. % to 95 wt. %, preferably from 1 wt. % to 90 wt. %, preferably from 1 wt. % to 80 wt. %, preferably from 1 wt. % to 50 wt. %, preferably from 1 wt. % to 25 wt. %, preferably from 1 wt. % to 20 wt. %, preferably from 1 wt. % to 10 wt. %, even more preferably from 1 wt. % to 5 wt. % based on the total weight of the composition.

According to any embodiments of the invention, the composition may comprise (+−)-(3E)-4-(2,6,6-trimethyl-1/2-cyclohexen-1-yl)-3-buten-2-one in an amount of at least 1 wt. %, preferably of at least 2 wt. %, even more preferably of at least 4 wt. %, based on the total weight of the composition. Particularly, the composition may comprise (+−)-(3E)-4-(2,6,6-trimethyl-1/2-cyclohexen-1-yl)-3-buten-2-one in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, preferably from 4 wt. % to 20 wt. %, even more preferably from 4 wt. % to 15 wt. % based on the total weight of the composition.

According to any embodiments of the invention, the composition may comprise 2-(3-phenylpropyl)pyridine in an amount of at least 0.0001 wt. %, preferably of at least 0.0005 wt. %, even more preferably of at least 0.0007 wt. %, based on the total weight of the composition. Particularly, the composition may comprise 2-(3-phenylpropyl)pyridine in an amount of from 0.0007 wt. % to 95 wt. %, preferably from 0.0007 wt. % to 90 wt. %, preferably from 0.0007 wt. % to 80 wt. %, preferably from 0.0007 wt. % to 50 wt. %, preferably from wt. % to 25 wt. %, preferably from 0.0007 wt. % to 20 wt. %, preferably from 0.0007 wt. % to 10 wt. %, preferably from 0.0007 wt. % to 5 wt. %, preferably from 0.0007 wt. % to 2 wt. %, preferably from 0.0007 wt. % to 1 wt. %, preferably from 0.0007 wt. % to 0.5 wt. %, preferably from 0.007 wt. % to 0.5 wt. %, preferably from 0.07 wt. % to 0.5 wt. %, even more preferably from 0.1 wt. % to 0.5 wt. % based on the total weight of the composition.

According to any embodiments of the invention, the composition may comprise (+−)-(2E)-1-(2,6,6-trimethyl-2-cyclohexen-1-yl)-2-buten-1-one in an amount of at least 0.1 wt. %, preferably of at least 0.3 wt. %, even more preferably of at least 0.5 wt. %, based on the total weight of the composition. Particularly, the composition may comprise (+−)-(2E)-1-(2,6,6-trimethyl-2-cyclohexen-1-yl)-2-buten-1-one in an amount of from 0.3 wt. % to 95 wt. %, preferably from 0.3 wt. % to 90 wt. %, preferably from 0.3 wt. % to 80 wt. %, preferably from wt. % to 50 wt. %, preferably from 0.3 wt. % to 25 wt. %, preferably from 0.3 wt. % to 20 wt. %, preferably from 0.3 wt. % to 10 wt. %, preferably from 0.3 wt. % to 5 wt. %, preferably from 0.3 wt. % to 2 wt. %, even more preferably from 0.3 wt. % to 1 wt. % based on the total weight of the composition.

According to any embodiments of the invention, the composition may comprise (+−)-6/8-sec-butylquinoline in an amount of at least 0.1 wt. %, preferably of at least 0.2 wt. %, even more preferably of at least 0.5 wt. %, based on the total weight of the composition. Particularly, the composition may comprise (+−)-6/8-sec-butylquinoline in an amount of from 0.2 wt. % to 95 wt. %, preferably from 0.2 wt. % to 90 wt. %, preferably from 0.2 wt. % to 80 wt. %, preferably from 0.2 wt. % to 50 wt. %, preferably from 0.2 wt. % to 25 wt. %, preferably from 0.2 wt. % to wt. %, preferably from 0.2 wt. % to 10 wt. %, preferably from 0.2 wt. % to 5 wt. %, even more preferably from 0.5 wt. % to 3 wt. % based on the total weight of the composition.

According to any embodiments of the invention, the composition may comprise (+−)-3,5,6,6-tetramethyl-4-methylidene-2-heptanone, (4E)-3,4,5,6,6-pentamethyl-4-hepten-2-one and 3,4,5,6,6-pentamethyl-3-hepten-2-one in an amount of at least 1 wt. %, preferably of at least 5 wt. %, even more preferably of at least 6 wt. %, based on the total weight of the composition. Particularly, the composition may comprise (+−)-3,5,6,6-tetramethyl-4-methylidene-2-heptanone, (4E)-3,4,5,6,6-pentamethyl-4-hepten-2-one and 3,4,5,6,6-pentamethyl-3-hepten-2-one, in an amount of from 5 wt. % to 95 wt. %, preferably from 5 wt. % to 90 wt. %, preferably from 5 wt. % to 80 wt. %, preferably from 5 wt. % to 50 wt. %, preferably from 5 wt. % to 25 wt. %, preferably from 5 wt. % to 20 wt. %, preferably from 5 wt. % to 10 wt. %, even more preferably from 6 wt. % to 10 wt. % based on the total weight of the composition.

According to any embodiments of the invention, the composition may comprise 1-(2-naphthyl)ethanone in an amount of at least 1 wt. %, preferably of at least 2 wt. %, even more preferably of at least 5 wt. %, based on the total weight of the composition. Particularly, the composition may comprise 1-(2-naphthyl)ethanone in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, preferably from 2 wt. % to 10 wt. %, even more preferably from 3 wt. % to 8 wt. % based on the total weight of the composition.

According to any embodiments of the invention, the composition may comprise methyl 2-((1RS,2RS)-3-oxo-2-pentylcyclopentyl)acetate in an amount of at least 5 wt. %, preferably of at least 10 wt. %, even more preferably of at least 50 wt. %, based on the total weight of the composition. Particularly, the composition may comprise methyl 2-((1RS,2RS)-3-oxo-2-pentylcyclopentyl)acetate in an amount of from 10 wt. % to 95 wt. %, preferably from 10 wt. % to 90 wt. %, preferably from 10 wt. % to 80 wt. %, preferably from 20 wt. % to 80 wt. %, preferably from 40 wt. % to 80 wt. %, even more preferably from 50 wt. % to 80 wt. %, based on the total weight of the composition.

According to any embodiments of the invention, the composition may comprise (2E,6Z)-2,6-nonadienal in an amount of at least 0.001 wt. %, preferably of at least 0.004 wt. %, even more preferably of at least 0.04 wt. %, based on the total weight of the composition. Particularly, the composition may comprise (2E,6Z)-2,6-nonadienal in an amount of from 0.004 wt. % to wt. %, preferably from 0.004 wt. % to 90 wt. %, preferably from 0.004 wt. % to 80 wt. %, preferably from 0.004 wt. % to 50 wt. %, preferably from 0.004 wt. % to 25 wt. %, preferably from 0.004 wt. % to 20 wt. %, preferably from 0.004 wt. % to 10 wt. %, preferably from 0.004 wt. % to 5 wt. %, preferably from 0.004 wt. % to 1 wt. %, preferably from 0.004 wt. % to 0.1 wt. %, even more preferably from 0.01 wt. % to 0.1 wt. %, based on the total weight of the composition.

According to any embodiments of the invention, the composition may comprise (+−)-(E)-4-methyl-3-decen-5-ol in an amount of at least 1 wt. %, preferably of at least 5 wt. %, even more preferably of at least 10 wt. %, based on the total weight of the composition. Particularly, the composition may comprise (+−)-(E)-4-methyl-3-decen-5-ol in an amount of from 5 wt. % to wt. %, preferably from 5 wt. % to 90 wt. %, preferably from 5 wt. % to 80 wt. %, preferably from 5 wt. % to 50 wt. %, preferably from 5 wt. % to 25 wt. %, preferably from 5 wt. % to 20 wt. %, even more preferably from 5 wt. % to 10 wt. %, based on the total weight of the composition.

According to any embodiments of the invention, the composition may comprise (3 aRS,5aSR,9aSR,9bRS)-3a,6,6,9a-tetramethyldodecahydronaphtho[2,1-b] furan in an amount of at least 1 wt. %, preferably of at least 2 wt. %, even more preferably of at least 10 wt. %, based on the total weight of the composition. Particularly, the composition may comprise (3 aRS,5aSR,9aSR,9bRS)-3a,6,6,9a-tetramethyldodecahydronaphtho[2,1-b] furan in an amount of from 1 wt. % to 95 wt. %, preferably from 1 wt. % to 90 wt. %, preferably from 1 wt. % to 80 wt. %, preferably from 1 wt. % to 50 wt. %, preferably from 1 wt. % to 25 wt. %, preferably from 1 wt. % to 20 wt. %, preferably from 1 wt. % to 10 wt. %, preferably from 1 wt. % to 5 wt. %, even more preferably from 2 wt. % to 5 wt. %, based on the total weight of the composition.

According to any embodiments of the invention, the composition may comprise 4-methoxybenzaldehyde in an amount of at least 1 wt. %, preferably of at least 2 wt. %, even more preferably of at least 10 wt. %, based on the total weight of the composition. Particularly, the composition may comprise 4-methoxybenzaldehyde in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, even more preferably from 10 wt. % to 20 wt. %, based on the total weight of the composition.

According to any embodiments of the invention, the composition may comprise 4-(4-hydroxyphenyl)-2-butanone in an amount of at least 1 wt. %, preferably of at least 3 wt. %, even more preferably of at least 7 wt. %, based on the total weight of the composition. Particularly, the composition may comprise 4-(4-hydroxyphenyl)-2-butanone in an amount of from 3 wt. % to 95 wt. %, preferably from 3 wt. % to 90 wt. %, preferably from 3 wt. % to 80 wt. %, preferably preferably from 3 wt. % to 50 wt. %, preferably from 3 wt. % to 25 wt. %, preferably from 3 wt. % to 20 wt. %, preferably from 3 wt. % to 10 wt. %, even more preferably from 7 wt. % to 10 wt. %, based on the total weight of the composition.

According to any embodiments of the invention, the composition may comprise tricyclo[5.2.1.0(2,6)]dec-3/4-en-8-yl propanoate in an amount of at least 1 wt. %, preferably of at least 3 wt. %, even more preferably of at least 10 wt. %, based on the total weight of the composition. Particularly, the composition may comprise tricyclo[5.2.1.0(2,6)]dec-3/4-en-8-yl propanoate in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 20 wt. %, even more preferably from 10 wt. % to 20 wt. %, based on the total weight of the composition.

According to any embodiments of the invention, the composition may comprise 2-chromenone in an amount of at least 1 wt. %, preferably of at least 5 wt. %, even more preferably of at least 9 wt. %, based on the total weight of the composition. Particularly, the composition may comprise 2-chromenone in an amount of from 9 wt. % to 95 wt. %, preferably from 9 wt. % to 90 wt. %, preferably from 9 wt. % to 80 wt %, preferably from 9 wt. % to 50 wt. %, preferably from 9 wt. % to 25 wt. %, preferably from 9 wt. % to 20 wt. %, even more preferably from 9 wt. % to 15 wt. %, based on the total weight of composition.

According to any embodiments of the invention, the composition may comprise (+−)-(3E)-3-methyl-4-(2,6,6-trimethyl-2-cyclohexen-1-yl)-3-buten-2-one and (+−)-(1E)-1-(2,6,6-trimethyl-2-cyclohexen-1-yl)-1-penten-3-one in an amount of at least 1 wt. %, preferably of at least 5 wt. %, even more preferably of at least 7 wt. %, based on the total weight of the composition. Particularly, the composition may comprise (+−)-(3E)-3-methyl-4-(2,6,6-trimethyl-2-cyclohexen-1-yl)-3-buten-2-one and (+−)-(1E)-1-(2,6,6-trimethyl-2-cyclohexen-1-yl)-1-penten-3-one in an amount of from 7 wt. % to 95 wt. %, preferably from 7 wt. % to 90 wt. %, preferably from 7 wt. % to 80 wt. %, preferably from 7 wt. % to 50 wt. %, even more preferably from 7 wt. % to 50 wt. %, based on the total weight of the composition.

According to any embodiments of the invention, the composition may comprise (+−)-(2,5-dimethyl-2,3-dihydro-1H-inden-2-yl)methanol in an amount of at least 1 wt. %, preferably of at least 4 wt. %, even more preferably of at least 10 wt. %, based on the total weight of the composition. Particularly, the composition may comprise (+−)-(2,5-dimethyl-2,3-dihydro-1H-inden-2-yl)methanol in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, preferably from 4 wt. % to 20 wt. %, preferably from 4 wt. % to 15 wt. %, even more preferably from 4 wt. % to 10 wt. %, based on the total weight of the composition.

According to any embodiments of the invention, the composition may comprise (E)-2-ethoxy-5-(1-propenyl)phenol in an amount of at least 0.005 wt. %, preferably of at least 0.05 wt. %, even more preferably of at least 0.5 wt. %, based on the total weight of the composition. Particularly, the composition may comprise (E)-2-ethoxy-5-(1-propenyl)phenol in an amount of from 0.005 wt. % to 95 wt. %, preferably from 0.005 wt. % to 90 wt. %, preferably from 0.005 wt. % to 80 wt. %, preferably from 0.005 wt. % to 50 wt. %, preferably from 0.005 wt. % to 25 wt. %, preferably from 0.005 wt. % to 20 wt. %, preferably from 0.005 wt. % to 15 wt. %, preferably from 0.005 wt. % to 10 wt. %, preferably from 0.05 wt. % to 10 wt. %, preferably from 0.5 wt. % to 10 wt. %, even more preferably from 1 wt. % to 10 wt. %, based on the total weight of the composition.

According to any embodiments of the invention, the composition may comprise undecanal in an amount of at least 0.05 wt. %, preferably of at least 0.5 wt. %, preferably of at least 1 wt. %, even more preferably of at least 3 wt. %, based on the total weight of the composition. Particularly, the composition may comprise undecanal in an amount of from 0.05 wt. % to 95 wt. %, preferably from 0.05 wt. % to 90 wt. %, preferably from 0.05 wt. % to 80 wt. %, preferably from 0.05 wt. % to 50 wt. %, preferably from 0.05 wt. % to 25 wt. %, preferably from 0.05 wt. % to 20 wt. %, preferably from 0.05 wt. % to 15 wt. %, preferably from 0.05 wt. % to 10 wt. %, preferably from 0.5 wt. % to 10 wt. %, preferably from 1 wt. % to 10 wt. %, even more preferably from 3 wt. % to 10 wt. %, based on the total weight of composition.

According to any embodiments of the invention, the composition may comprise 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone and 1-[(2RS,3RS,8aRS)-2,3,8,8-tetramethyl-1,2,3,4,6,7,8,8a-octahydro-2-naphthalenyl]ethanone in an amount of at least 1 wt. %, preferably of at least 5 wt. %, preferably of at least 8 wt. %, even more preferably of at least 10 wt. %, based on the total weight of the composition. Particularly, the composition may comprise 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone and 1-[(2RS,3RS,8aRS)-2,3,8,8-tetramethyl-1,2,3,4,6,7,8,8a-octahydro-2-naphthalenyl]ethanone in an amount of from 8 wt. % to 95 wt. %, preferably from 8 wt. % to 90 wt. %, preferably from 8 wt. % to 80 wt. %, preferably from 8 wt. % to 50 wt. %, even more preferably from 8 wt. % to wt. %, based on the total weight of the composition.

According to any embodiments of the invention, the composition may comprise (+−)-2,6-dimethyl-7-octen-2-ol in an amount of at least 10 wt. %, preferably of at least 50 wt. %, even more preferably of at least 70 wt. %, based on the total weight of the composition. Particularly, the composition may comprise (+−)-2,6-dimethyl-7-octen-2-ol in an amount of from 10 wt. % to 95 wt. %, preferably from 20 wt. % to 95 wt. %, preferably from 30 wt. % to 95 wt. %, preferably from 40 wt. % to 95 wt. %, preferably from 50 wt. % to 95 wt. %, preferably from 60 wt. % to 95 wt. %, even more preferably from 70 wt. % to 95 wt. %, based on the total weight of the composition.

According to any embodiments of the invention, the composition may comprise 2-phenylethanol in an amount of at least 3 wt. %, preferably of at least 10 wt. %, even more preferably of at least 20 wt. %, based on the total weight of the composition. Particularly, the composition may comprise 2-phenylethanol in an amount of from 3 wt. % to 95 wt. %, preferably from 3 wt. % to 90 wt. %, preferably from 3 wt. % to 80 wt. %, preferably from 3 wt. % to 50 wt. %, preferably from 10 wt. % to 50 wt. %, even more preferably from 20 wt. % to wt. %, based on the total weight of the composition.

According to any embodiments of the invention, the composition may comprise (3Z)-3-hexen-1-ol in an amount of at least 0.1 wt. %, preferably of at least 0.4 wt. %, even more preferably of at least 1 wt. %, based on the total weight of the composition. Particularly, the composition may comprise (3Z)-3-hexen-1-ol in an amount of from 0.4 wt. % to 95 wt. %, preferably from 0.4 wt. % to 90 wt. %, preferably from 0.4 wt. % to 80 wt. %, preferably from 0.4 wt. % to 50 wt. %, preferably from 0.4 wt. % to 25 wt. %, preferably from 0.4 wt. % to 20 wt. %, preferably from 0.4 wt. % to 10 wt. %, preferably from 1 wt. % to 10 wt %, even more preferably from 2 wt. % to 8 wt. % based on the total weight of the composition.

According to any embodiments of the invention, the composition may comprise (4Z)-4-dodecenal in an amount of at least 0.01 wt. %, preferably of at least 0.1 wt. %, even more preferably of at least 0.3 wt. %, based on the total weight of the composition. Particularly, the composition may comprise (4Z)-4-dodecenal in an amount of from 0.01 wt. % to 95 wt. %, preferably from 0.01 wt. % to 90 wt. %, preferably from 0.01 wt. % to 80 wt. %, preferably from 0.01 wt. % to 50 wt. %, preferably from 0.01 wt. % to 25 wt. %, preferably from 0.01 wt. % to 20 wt. %, preferably from 0.01 wt. % to 10 wt. %, preferably from 0.01 wt. % to 5 wt %, preferably from 0.01 wt. % to 1 wt. %, even more preferably from 0.1 wt. % to 1 wt. % based on the total weight of the composition.

According to any embodiments of the invention, the composition may comprise octanal in an amount of at least 1 wt. %, preferably of at least 3 wt. %, even more preferably of at least 4 wt. %, based on the total weight of the composition. Particularly, the composition may comprise octanal in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 20 wt. %, preferably from 3 wt. % to 15 wt. %, preferably from 3 wt. % to 10 wt. %, even more preferably from 4 wt. % to 10 wt. %, based on the total weight of the composition.

According to any embodiments of the invention, the composition may comprise decanal in an amount of at least 1 wt. %, preferably of at least 5 wt. %, even more preferably of at least 10 wt. %, based on the total weight of the composition. Particularly, the composition may comprise decanal in an amount of from 5 wt. % to 95 wt. %, preferably from 5 wt. % to 90 wt. %, preferably from 5 wt. % to 80 wt. %, preferably from 5 wt. % to 50 wt. %, preferably from 5 wt. % to 25 wt. %, preferably from 5 wt. % to 20 wt. %, even more preferably from 1 wt. % to 20 wt. %, based on the total weight of the composition.

According to any embodiments of the invention, the composition may comprise 10-undecenal in an amount of at least 0.1 wt. %, preferably of at least 0.4 wt. %, even more preferably of at least 1 wt. %, based on the total weight of the composition. Particularly, the composition may comprise 10-undecenal in an amount of from 0.4 wt. % to 95 wt. %, preferably from 0.4 wt. % to 90 wt. %, preferably from 0.4 wt. % to 80 wt. %, preferably from 0.4 wt. % to 50 wt. %, preferably from 0.4 wt. % to 25 wt. %, preferably from 0.4 wt. % to 20 wt. %, preferably from wt. % to 10 wt. %, preferably from 0.4 wt. % to 5 wt %, even more preferably from 1 wt. % to 5 wt. % based on the total weight of the composition.

According to any embodiments of the invention, the composition may comprise 3-ethoxy-4-hydroxybenzaldehyde in an amount of at least 1 wt. %, preferably of at least 2 wt. %, even more preferably of at least 10 wt. %, based on the total weight of the composition. Particularly, the composition may comprise 3-ethoxy-4-hydroxybenzaldehyde in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, preferably from 5 wt. % to 20 wt. %, even more preferably from 10 wt. % to 20 wt. %, based on the total weight of the composition.

According to any embodiments of the invention, the composition may comprise 2-ethyl-3-hydroxy-4(4H)-pyranone in an amount of at least 1 wt. %, preferably of at least 6 wt. %, even more preferably of at least 10 wt. %, based on the total weight of the composition. Particularly, the composition may comprise 2-ethyl-3-hydroxy-4(4H)-pyranone in an amount of from 1 wt. % to 95 wt. %, preferably from 1 wt. % to 90 wt. %, preferably from 1 wt. % to 80 wt. %, preferably from 1 wt. % to 50 wt. %, preferably from 1 wt. % to 25 wt. %, preferably from 1 wt. % to 20 wt. %, preferably from 1 wt. % to 10 wt. %, even more preferably from 6 wt. % to 10 wt. %, based on the total weight of the composition.

According to any embodiments of the invention, the composition may comprise (E)-3,7-dimethyl-2,6-octadien-1-ol in an amount of at least 1 wt. %, preferably of at least 4 wt. %, even more preferably of at least 10 wt. %, based on the total weight of the composition. Particularly, the composition may comprise (E)-3,7-dimethyl-2,6-octadien-1-ol in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, preferably from 4 wt. % to 20 wt. %, even more preferably from 10 wt. % to 20 wt. %, based on the total weight of the composition.

According to any embodiments of the invention, the composition may comprise (+−)-tetrahydro-2-isobutyl-4-methyl-4(2H)-pyranol in an amount of at least 1 wt. %, preferably of at least 9 wt. %, even more preferably of at least 20 wt. %, based on the total weight of the composition. Particularly, the composition may comprise (+−)-tetrahydro-2-isobutyl-4-methyl-4(2H)-pyranol in an amount of from 9 wt. % to 95 wt. %, preferably from 9 wt. % to 90 wt. %, preferably from 9 wt. % to 80 wt. %, preferably from 9 wt. % to 50 wt. %, preferably from 15 wt. % to 50 wt. %, even more preferably from 20 wt. % to 50 wt. %, based on the total weight of the composition.

According to any embodiments of the invention, the composition may comprise (1RS,2RS)-2,4-dimethyl-3-cyclohexene-1-carbaldehyde and (1RS,2 SR)-2,4-dimethyl-3-cyclohexene-1-carbaldehyde in an amount of at least 1 wt. %, preferably of at least 2 wt. %, even more preferably of at least 10 wt. %, based on the total weight of the composition. Particularly, the composition may comprise (1RS,2RS)-2,4-dimethyl-3-cyclohexene-1-carbaldehyde and (1RS,2SR)-2,4-dimethyl-3-cyclohexene-1-carbaldehyde in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, even more preferably from 2 wt. % to 10 wt. %, based on the total weight of the composition.

According to any embodiments of the invention, the composition may comprise (+−)-3/4-(4-methyl-3-penten-1-yl)-3-cyclohexene-1-carbaldehyde in an amount of at least 0.01 wt. %, preferably of at least 0.1 wt. %, preferably of at least 0.5 wt. %, even more preferably of at least 5 wt. %, based on the total weight of the composition. Particularly, the composition may comprise (+−)-3/4-(4-methyl-3-penten-1-yl)-3-cyclohexene-1-carbaldehyde in an amount of from 0.5 wt. % to 95 wt. %, preferably from 0.5 wt. % to 90 wt. %, preferably from 0.5 wt. % to wt. %, preferably from 0.5 wt. % to 50 wt. %, preferably from 0.5 wt. % to 25 wt. %, preferably from 0.5 wt. % to 20 wt. %, preferably from 0.5 wt. % to 10 wt. %, even more preferably from 1 wt. % to 10 wt. %, based on the total weight of the composition.

In some aspects, the amount sufficient to reduce, prevent, or inhibit the subject's perception of sweat malodor is from 10 to 100 ppm.

In some aspects, the amount sufficient to reduce, prevent, or inhibit the subject's perception of sweat malodor is from 20 to 100 ppm. In some aspects, the amount sufficient to reduce, prevent, or inhibit the subject's perception of sweat malodor is from 30 to 100 ppm. In some aspects, the amount sufficient to reduce, prevent, or inhibit the subject's perception of sweat malodor is from 40 to 100 ppm. In some aspects, the amount sufficient to reduce, prevent, or inhibit the subject's perception of sweat malodor is from 50 to 100 ppm. In some aspects, the amount sufficient to reduce, prevent, or inhibit the subject's perception of sweat malodor is from 60 to 100 ppm. In some aspects, the amount sufficient to reduce, prevent, or inhibit the subject's perception of sweat malodor is from 70 to 100 ppm. In some aspects, the amount sufficient to reduce, prevent, or inhibit the subject's perception of sweat malodor is from 80 to 100 ppm. In some aspects, the amount sufficient to reduce, prevent, or inhibit the subject's perception of sweat malodor is from 90 to 100 ppm.

In some aspects, the amount sufficient to reduce, prevent, or inhibit the subject's perception of sweat malodor is from 10 to 90 ppm. In some aspects, the amount sufficient to reduce, prevent, or inhibit the subject's perception of sweat malodor is from 10 to 80 ppm. In some aspects, the amount sufficient to reduce, prevent, or inhibit the subject's perception of sweat malodor is from 10 to 70 ppm. In some aspects, the amount sufficient to reduce, prevent, or inhibit the subject's perception of sweat malodor is from 10 to 60 ppm. In some aspects, the amount sufficient to reduce, prevent, or inhibit the subject's perception of sweat malodor is from 10 to 50 ppm. In some aspects, the amount sufficient to reduce, prevent, or inhibit the subject's perception of sweat malodor is from 10 to 40 ppm. In some aspects, the amount sufficient to reduce, prevent, or inhibit the subject's perception of sweat malodor is from 10 to 30 ppm. In some aspects, the amount sufficient to reduce, prevent, or inhibit the subject's perception of sweat malodor is from 10 to 20 ppm.

In one aspect a compound of the present invention may be comprised about 0.01 to about 100 wt % of the total weight of the composition. In further aspects, the compounds may be comprised: about 0.02 to about 100 wt %, about 0.03 to about 100 wt %, about 0.04 to about 100 wt %, about 0.05 to about 100 wt %, about 0.06 to about 100 wt %, about 0.07 to about 100 wt %, about 0.08 to about 100 wt %, about 0.09 to about 100 wt %, about 0.1 to about 100 wt %, about 0.2 to about 100 wt %, about 0.3 to about 100 wt %, about 0.4 to about 100 wt %, about 0.5 to about 100 wt %, about 0.6 to about 100 wt %, about 0.7 to about 100 wt %, about 0.8 to about 100 wt %, about 0.9 to about 100 wt %, about 1.0 to about 100 wt %, about 1.1 to about 100 wt %, about 1.2 to about 100 wt %, about 1.3 to about 100 wt %, about 1.4 to about 100 wt %, about 1.5 to about 100 wt %, about 1.6 to about 100 wt %, about 1.7 to about 100 wt %, about 1.8 to about 100 wt %, about 1.9 to about 100 wt %, about 2 to about 100 wt %, about 3 to about 100 wt %, about 4 to about 100 wt %, about 5 to about 100 wt %, about 6 to about 100 wt %, about 7 to about 100 wt %, about 8 to about 100 wt %, about 9 to about 100 wt %, about 10 to about 100 wt %, about 11 to about 100 wt %, about 12 to about 100 wt %, about 13 to about 100 wt %, about 14 to about 100 wt %, about 15 to about 100 wt %, about 16 to about 100 wt %, about 17 to about 100 wt %, about 18 to about 100 wt %, and about 20 to about 100 wt % of the total weight of the composition.

In one aspect a compound of the present invention may be comprised about 0.01 to about 99 wt % of the total weight of the composition. In further aspects, the compounds may be comprised: about 0.02 to about 99 wt %, about 0.03 to about 99 wt %, about 0.04 to about 99 wt %, about 0.05 to about 99 wt %, about 0.06 to about 99 wt %, about 0.07 to about 99 wt %, about 0.08 to about 99 wt %, about 0.09 to about 99 wt %, about 0.1 to about 99 wt %, about 0.2 to about 99 wt %, about 0.3 to about 99 wt %, about 0.4 to about 99 wt %, about 0.5 to about 99 wt %, about 0.6 to about 99 wt %, about 0.7 to about 99 wt %, about 0.8 to about 99 wt %, about 0.9 to about 99 wt %, about 1.0 to about 99 wt %, about 1.1 to about 99 wt %, about 1.2 to about 99 wt %, about 1.3 to about 99 wt %, about 1.4 to about 99 wt %, about 1.5 to about 99 wt %, about 1.6 to about 99 wt %, about 1.7 to about 99 wt %, about 1.8 to about 99 wt %, about 1.9 to about 99 wt %, about 2 to about 99 wt %, about 3 to about 99 wt %, about 4 to about 99 wt %, about 5 to about 99 wt %, about 6 to about 99 wt %, about 7 to about 99 wt %, about 8 to about 99 wt %, about 9 to about 99 wt %, about 10 to about 99 wt %, about 11 to about 99 wt %, about 12 to about 99 wt %, about 13 to about 99 wt %, about 14 to about 99 wt %, about 15 to about 99 wt %, about 16 to about 99 wt %, about 17 to about 99 wt %, about 18 to about 99 wt %, and about 20 to about 99 wt % of the total weight of the composition.

In one aspect a compound of the present invention may be comprised about 0.01 to about 95 wt % of the total weight of the composition. In further aspects, the compounds may be comprised: about 0.02 to about 95 wt %, about 0.03 to about 95 wt %, about 0.04 to about 95 wt %, about 0.05 to about 95 wt %, about 0.06 to about 95 wt %, about 0.07 to about 95 wt %, about 0.08 to about 95 wt %, about 0.09 to about 95 wt %, about 0.1 to about 95 wt %, about to about 95 wt %, about 0.3 to about 95 wt %, about 0.4 to about 95 wt %, about 0.5 to about 95 wt %, about 0.6 to about 95 wt %, about 0.7 to about 95 wt %, about 0.8 to about 95 wt %, about 0.9 to about 95 wt %, about 1.0 to about 95 wt %, about 1.1 to about 95 wt %, about 1.2 to about 95 wt %, about 1.3 to about 95 wt %, about 1.4 to about 95 wt %, about 1.5 to about 95 wt %, about 1.6 to about 95 wt %, about 1.7 to about 95 wt %, about 1.8 to about wt %, about 1.9 to about 95 wt %, about 2 to about 95 wt %, about 3 to about 95 wt %, about 4 to about 95 wt %, about 5 to about 95 wt %, about 6 to about 95 wt %, about 7 to about 95 wt %, about 8 to about 95 wt %, about 9 to about 95 wt %, about 10 to about 95 wt %, about 11 to about 95 wt %, about 12 to about 95 wt %, about 13 to about 95 wt %, about 14 to about 95 wt %, about 15 to about 95 wt %, about 16 to about 95 wt %, about 17 to about 95 wt %, about 18 to about 95 wt %, and about 20 to about 95 wt % of the total weight of the composition.

In some aspects, the term “contacting” refers to administering to a subject, a composition comprising the at least one compound as described herein, wherein the administering results in dosing the subject with an effective amount of the at least one compound. Administration may be via any method readily selected by one of ordinary skill in the art. Methods include, but are not limited to, topical administration, and inhalation. Accordingly the present disclosure contemplates formulating a composition comprising the at least one compound as described herein with a suitable carrier to facilitate administering a composition comprising the at least one compound as described herein to the subject.

In some aspects, the term “contacting” refers to dispensing or dispersing a composition comprising the at least one compound as described herein into a volume in need thereof, wherein the dispensing or dispersing results in dosing the subject with an effective amount of the at least one compound. Dispersion or dispensing of the at least one compound as described herein may be achieved by any method readily selected by one of ordinary skill in the art. Examples include, but are not limited to, a spray, a nebulizer, evaporation of a solution containing the at least one compound as described herein, and the like.

Accordingly the present disclosure contemplates formulating a composition comprising the at least one compound as described herein with a suitable carrier to facilitate treating a surface or volume with a composition comprising the at least one compound as described herein to the subject.

In some aspects, the term “contacting” refers to contacting a surface of a sweat malodor source with a composition comprising the at least one compound as described herein, wherein the contacting results in an effective amount of the at least one compound being deposited on the surface. A composition comprising the at least one compound as described herein may be contacted on a surface by any method known in the art. Examples include, but are not limited to, a spray, a wipe, a solution, and the like.

Products and Formulations According to Some Aspects Presented Herein

It is understood by a person skilled in the art that the at least one compound, as defined herein, may be added into a composition described herein in neat form, or in a solvent. Alternatively, the at least one compound may first be modified, for example by entrapped with an entrapment material such as for example polymers, capsules, microcapsules, nanocapsules, liposomes, precursors, film formers, absorbents such as for example by using carbon or zeolites, cyclic oligosaccharides and mixtures thereof. Alternatively, the at least one compound may be chemically bound to substrates which are adapted to release the compounds upon application of an exogenous stimulus such as light, enzymes, or the like.

Accordingly, some aspects presented herein provide a composition comprising:
a. the at least one compound;
b. at least one ingredient selected from the group consisting of a perfumery carrier and a perfumery base; and
c. optionally at least one perfumery adjuvant.

As used herein, the term “perfumery carrier” refers to a material which is practically neutral from a perfumery point of view, i.e. which does not significantly alter the organoleptic properties of perfuming ingredients. The perfumery carrier may be a liquid or a solid.

A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethanol, tri-ethyl citrate or mixtures thereof, which are the most commonly used. For the compositions which comprise both a perfumery carrier and a perfumery base, other suitable perfumery carriers may include ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company) or hydrogenated castors oils such as those known under the trademark Cremophor® RH 40 (origin: BASF).

Solid carrier is meant to designate a material to which the composition or some element of the composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- and Geliermittel in Lebensmitteln, Band 2 der Schriftenreihe Lebensmittelchemie, Lebensmittelqualitat, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

As used herein, the term “perfumery base” refers a composition comprising at least one perfume ingredient. A perfume ingredient does not include the at least one compound. As used herein, the term “perfume ingredient” refers to a perfume compound, which is used in a perfuming preparation or a composition to impart a hedonic effect. In other words such a perfume ingredient, to be considered as being a perfuming one, must be recognized by a person skilled in the art as being able to impart or modify in a positive or pleasant way the odor of a composition.

In general terms, perfume ingredients belong to chemical classes as varied as alcohols, lactones, aldehydes, ketones, esters, ethers, acetates, nitriles, terpenoids, nitrogenous or sulphurous heterocyclic compounds and essential oils, and the perfume ingredients can be of natural or synthetic origin. Many of these perfume ingredients are listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that the perfume ingredients may also be compounds known to release in a controlled manner, also known as properfume or profragrance. Non-limiting examples of suitable properfume may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo (phenyl) acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-44(2-methoxy-2-phenylvinyl)oxy)benz aldehyde, 4-((2-(hexyloxy)-2-phenylvinyl)oxy)-3-methoxybenzaldehyde or a mixture thereof.

Perfuming ingredients which are commonly used in perfume formulations include:
Aldehydic ingredients: decanal, dodecanal, 2-methyl-undecanal, 10-undecenal, octanal and/or nonenal;
Aromatic-herbal ingredients: eucalyptus oil, camphor, eucalyptol, menthol and/or alpha-pinene;
Balsamic ingredients: coumarine, ethylvanillin and/or vanillin;
Citrus ingredients: dihydromyrcenol, 3,7-dimethylocta-2,6-dienal, orange oil, linalyl acetate, (−)-(R)-3,7-dimethyl-6-octenenitrile, orange terpenes, limonene, 1-P-menthen-8-yl acetate and/or 1,4(8)-p-menthadiene;
Floral ingredients: Methyl dihydrojasmonate, linalool, Citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, Propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma undecalactone, 4-decanolide, ethyl 2-methyl-pentanoate, hexyl acetate, ethyl 2-methylbutanoate, gamma nonalactone, allyl heptanoate, 2-phenoxyethyl isobutyrate, ethyl 2-methyl-1,3-dioxolane-2-acetate and/or diethyl 1,4-cyclohexane dicarboxylate;
Green ingredients: 2,4-Dimethyl-3-cyclohexene-1-carbaldehyde, 2-tert-butyl-1-cyclohexyl acetate, (+−)-1-phenylethyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, pentadecenolide, 3-Methyl-5-cyclopentadecen-1-one, 1,3,4,6,7,8-hexahydro-4,6,6,7,8,8-hexamethyl-(1S,1′R)-2-[1-(3′,3′-dimethyl-1′-cyclohexyl)ethoxy]-2-methylpropyl propanoate, pentadecanolide and/or (1S,1′R)-[1-(3′,3′-Dimethyl-1′-cyclohexyl)ethoxycarbonyl]methyl propanoate;
Woody ingredients: 1-(octahydro-2,3,8,8-tetramethyl-2-naphtalenyl)-1-ethanone, patchouli oil, terpenes fractions of patchouli oil, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, Methyl cedryl ketone, 5-(2,2,3-trimethyl-3-cyclopentenyl)-3-methylpentan-2-ol, 1−(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal and/or 3-(3-isopropyl-1-phenyl)butanal.

As used herein, the term “perfumery adjuvant” refers to an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

As used herein, the term perfume or perfume oil or perfume accord are used to designate a mixture of perfuming ingredients.

Delivery System: Compositions according to the present invention can be used in combination with a delivery system; i.e. the invention's composition or some elements may be encapsulated or the invention's composition may further comprise a delivery system. Suitable delivery systems for the purpose of the present disclosure include but are not limited to:
Passive plating supports comprising one or more of the following porous or non-porous substrates in loose powder or compacted form chosen from the following non-limiting examples: cellulose (paper/cardboard), vermiculite, other industrial absorbents, perlite, calcium carbonate, pumice, other minerals, wood, sawdust, ground corn cob, ground rice hull, rice hull ash, other agricultural by-products, biochars, starches, modified starches;
Spray-dried moisture-activated encapsulation systems wherein compositions according to the present disclosure are encapsulated by a spray drying process within a matrix containing but not limited to one or more of the following: maltodextrin, octenyl succinated starch (modified starch);
Core-shell encapsulation systems, such as mechanically activated microcapsules with an impermeable shell (for example, polyurea, polyurethane, and others) and composition according to the present disclosure in the core;
Liquid mixtures containing surfactants;
Polymeric materials.

The present invention encompasses the composition combined with encapsulating materials such as polymers to form microcapsules or microparticles, or materials to form liquid delivery system for the composition such as an emulsion, a microemulsion, a miniemulsion, a gel, a microgel, an anhydrous gel or a dispersion.

According to a particular aspect, the composition is absorbed on a porous or non-porous substrate in loose powder or compacted form, the substrate being selected from cellulose (paper/cardboard), vermiculite, other industrial absorbents, perlite, calcium carbonate, pumice, wood, sawdust, ground corn cob, ground rice hull, rice hull ash, biochars, starches, modified starches and mixtures thereof.

In some aspects, the composition further comprises at least one other maolodor control (MOC) compound. As used herein, the term “other MOC compound” refers to a material which is already known for a MOC activity and is commonly used in the industry for such use. The at least one other MOC compound can be included to further boost, or complement, the MOC activity of a compound of the present invention.

Non-limiting examples of an other MOC compound include antimicrobial agents, malodor absorbers, chemical neutralisers e.g. acid-base reagents, thiol traps, odor blockers, cross-adaptation agents e.g. as disclosed in U.S. Pat. No. 5,538,719 incorporated herein by reference, and malodor complexation agents, e.g. cyclodextrins.

Examples of antimicrobial agents include, but are not limited to, metal salts such as zinc citrate, zinc oxide, zinc pyrethiones, and octopirox; organic acids, such as sorbic acid, benzoic acid, and their salts; parabens, such as methyl paraben, propyl paraben, butyl paraben, ethyl paraben, isopropyl paraben, isobutyl paraben, benzyl paraben, and their salts; alcohols, such as benzyl alcohol, phenyl ethyl alcohol; boric acid; 2,4,4′-trichloro-2-hydroxy-diphenyl ether; phenolic compounds, such as phenol, 2-methyl phenol, 4-ethyl phenol; essential oils such as rosemary, thyme, lavender, eugenol, geranium, tea tree, clove, lemon grass, peppermint, or their active components such as anethole, thymol, eucalyptol, farnesol, menthol, limonene, methyl salicylate, salicylic acid, terpineol, nerolidol, geraniol, and mixtures thereof.

Examples of malodor absorbers include, but are not limited to molecular sieves, such as zeolites, silicas, aluminosilcates, and cyclodextrins; and organic absorbents, such as for example, activated charcoal, dried citrus pulp, cherry pit extract, corncob, and mixtures thereof.

In some aspects, the present disclosure provides a perfumed consumer product comprising the at least one compound in an amount sufficient to reduce, prevent, or inhibit the subject's perception of malodor.

In some aspects, the consumer product comprising an effective amount of the at least one compound and a perfume ingredient.

Non-limiting examples of consumer products according to the present invention include:
a perfume, such as a fine perfume, an Eau de Toilette, a cologne or an after-shave lotion;
a hair care product, such as a shampoo, a hair conditioner, a hair cream, a hair oil, a hair styling product (such as a spray, mousse or gel), a hair coloration product or a hair permanent wave product;
a skin care product, such as a face cream, a face lotion, a shaving product (such as a foam, cream, gel or oil), a body and/or hand product (such as a lotion, cream, gel or oil), a skin firming product, a depilatory, a talcum powder, a foot care cream or lotion, baby wipes, cleansing wipes, moisturizer wipes, a sun-protection product (such as a spray, lotion, cream or oil), an after-sun lotion, or a self-tanning product;
a fabric care product, such as a liquid detergent, a powder detergent, detergent tablets, a detergent bar, a detergent paste, a detergent pouch, a liquid fabric softener, fabric softener sheets, a fabric scent booster, a laundry pre-treatment, a fabric refresher, an ironing water, a laundry bleach, a carpet powder or a carpet cleaner;
a body deodorant or antiperspirant product, such as a body deodorant spray, a roll-on deodorant, a deodorant stick, a deodorant cream, an antiperspirant spray, an antiperspirant stick, a roll-on antiperspirant liquid, an antiperspirant stick, or an antiperspirant cream;
a skin-cleansing product, such as a soap bar, a shower gel, a liquid hand soap, a bath foam or an intimate wash product; and
an air freshening product, such as an air freshener spray, a gel air freshener, a liquid-wick air freshener, a solid air freshener comprising a porous substrate (such as a paper or card blotter, a porous ceramic, or a porous plastic), a liquid or gel air freshener comprising a permeable membrane, an electrically operated air freshener, and a dual purpose air freshener/disinfectant spray.

Accordingly, in some aspects, the present disclosure provides a consumer product comprising an effective amount of the at least one compound.

In some aspects, the consumer product comprising an effective amount of the compound comprises a formulation selected from the group consisting of: aerosol and/or water-based air freshener spray, wick/reed air freshener, liquid electrical (plug-in) air freshener, a solid support air freshener, gel-based air freshener, membrane-containing air freshener, bleaching, cleaning, liquid all-purpose cleaner, specialty cleaner and liquid detergent.

One of ordinary skill in the art would appreciate that the conception and the specific aspects disclosed might be readily utilized as a basis for modifying or formulating other formulations for carrying the same purposes of the present invention. It should also be realized by those skilled in the art that such equivalent formulations do not depart from the spirit and scope of the disclosure as set forth herein.

The concentration of MOC composition according to the aspects described herein, used in the various aforementioned consumer products varies within a various wide range of values depending on the nature of the consumer product. For instance, a MOC composition according some aspects described herein can be used in a perfumed product at a concentration of 0.01% to 50% by weight, alternatively at a concentration of 0.2% to 40% by weight, alternatively at a concentration of 0.5% to 25% by weight. For instance, a MOC composition according to some aspects described herein can be used in a fabric care product at a concentration of 0.01% to 20% by weight, alternatively at a concentration of 0.05% to 10% by weight, alternatively at a concentration of 0.1% to 5% by weight. Yet for instance, a MOC composition according to some aspects described herein can be used in a hair care product at a concentration of 0.01% to 10% by weight, alternatively at a concentration of to 5% by weight, alternatively at a concentration of 0.1% to 3% by weight. For instance, a MOC composition according to some aspects described herein can be used in a skin care product at a concentration of 0.01% to 10% by weight, alternatively at a concentration of 0.05% to 5% by weight, most preferably at a concentration of 0.1% to 2.5% by weight. For instance, a MOC composition according to some aspects described herein can be used in a body deodorant or antiperspirant product at a concentration of 0.01% to 10% by weight, alternatively at a concentration of 0.05% to 7% by weight, alternatively at a concentration of 0.1% to 5% by weight. Yet for instance, a MOC composition according to some aspects described herein can be used in a skin cleansing product at a concentration of to 5% by weight, alternatively at a concentration of 0.05% to 3% by weight, alternatively at a concentration of 0.1% to 2.5% by weight. For instance, a MOC composition according to some aspects described herein can be used in an air freshening product at a concentration of 0.01% to 100% by weight. For instance, a MOC composition according to some aspects described herein can be used in a surface care product at a concentration of 0.001% to 10% by weight, alternatively at a concentration of 0.01% to 5% by weight, alternatively at a concentration of 0.1% to 2% by weight.

In some aspects, the perfumed consumer product is selected from the group consisting of: an anti-perspirant, a deodorant, deodorant spray, a roll-on deodorant, a deodorant stick, a deodorant cream, an antiperspirant spray, an antiperspirant stick, a roll-on antiperspirant liquid, an antiperspirant stick, or an antiperspirant cream.

As used herein, the term “antiperspirant or deodorant product” refers to the normal meaning in the art; i.e. a composition applied on skin allowing to reduce or prevent body odour. Particularly, the perfumed consumer product may be in is in the form of a body deodorant spray, a roll-on deodorant, a deodorant stick, a deodorant cream, an antiperspirant spray, an antiperspirant stick, a roll-on antiperspirant liquid, an antiperspirant stick, or an antiperspirant cream. The perfumed consumer product may be in is in the form of a deodorant or antiperspirant product comprising at least one deodorant or antiperspirant active base. Non-limiting examples of suitable deodorant or antiperspirant active base may include emollients, solubilizers, deodorant actives, antioxidants, preservatives, carriers, odour entrappers, propellants, primary structurants, antiperspirant actives, additional chassis ingredients, volatile silicone solvents, gellants, buffering agent and residue masking materials. A person skilled in the art is able to select them on the basis of its general knowledge and according to intended form of the deodorant or antiperspirant composition.

The deodorant or antiperspirant may be in the form of wax-based sticks, soap-based sticks, compressed powder sticks, roll-on suspensions or solutions, emulsions, gels, creams, squeeze sprays, pump sprays, aerosols, and the like. Each product form may contain its own selection of additional deodorant or antiperspirant active base, some essential and some optional. For example, by way of illustration, a roll-on deodorant or antiperspirant product may comprise water, emollient, solubilizer, deodorant or antiperspirant actives, antioxidants, preservatives, or combinations thereof; a clear gel product or antiperspirant product may comprise water, emollient, solubilizer, deodorant or antiperspirant actives, antioxidants, preservatives, ethanol, or combinations thereof; a body spray may contain a carrier, deodorant or antiperspirant actives, odour entrappers, propellant, or combinations thereof; an invisible solid deodorant or antiperspirant product may contain a primary structurant, deodorant or antiperspirant actives, and additional chassis ingredient(s); a soft solid deodorant or antiperspirant product may comprise volatile silicone, deodorant or antiperspirant actives, gellant, residue masking material, or combinations thereof; an aerosol deodorant or antiperspirant product may comprise a carrier, a propellant, or a combination thereof.

Emollients suitable for deodorant or antiperspirant products include, but are not limited to, propylene glycol, polypropylene glycol (like dipropylene glycol, tripropylene glycol, etc.), diethylene glycol, triethylene glycol, neopentyl glycol diheptanoate, PEG-4, PEG-8, 1,2-pentanediol, 1,2-hexanediol, hexylene glycol, glycerin, C₂ to C₂₀ monohydric alcohols, C₂ to C₄₀ dihydric or polyhydric alcohols, alkyl ethers of polyhydric and monohydric alcohols, dicaprylyl carbonate, dicaprylyl ether, diethylhexylcyclohexane, dibutyl adipate, volatile silicone emollients such as cyclopentasiloxane, nonvolatile silicone emollients such as dimethicone, mineral oils, polydecenes, petrolatum, and combinations thereof. One example of a suitable emollient comprises PPG-15 stearyl ether. Other examples of suitable emollients include dipropylene glycol and propylene glycol.

Suitable deodorant actives can include any topical material that is known or otherwise effective in preventing or eliminating malodour, including malodour associated with sweat and/or perspiration. Suitable deodorant actives may be selected from the group consisting of antimicrobial agents (e.g., bacteriocides, fungicides), malodour-absorbing material, and combinations thereof. Antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′hydroxy diphenyl ether (triclosan), 3,4,4′-trichlorocarbanilide (triclocarban), diaminoalkyl amides such as L®-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. Suitable odour entrappers for use herein include, for example, solubilized, water-soluble, uncomplexed cyclodextrin. As used herein, the term “cyclodextrin” includes any of the known cyclodextrins such as unsubstituted cyclodextrins containing from six to twelve glucose units, including alpha-cyclodextrin, beta-cyclodextrin, gamma-cyclodextrin and/or their derivatives and/or mixtures thereof.

A suitable solubilizer for deodorant or antiperspirant products can be, for example, a surfactant, such as a no-foaming or low-foaming surfactant. Suitable surfactants are nonionic surfactants, cationic surfactants, amphoteric surfactants, zwitterionic surfactants, and mixtures thereof. Suitable solubilizers include, for example, polyethylene glycol ether of Cetearyl Alcohol, hydrogenated castor oil such as polyoxyethylene hydrogenated castor oil, polyoxyethylene 2 stearyl ether, polyoxyethylene 20 stearyl ether, and combinations thereof. Suitable preservatives for deodorant or antiperspirant products include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof. Non-limiting examples of commercially available preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathan® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis {N′-[l-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, German 115C) from Sutton Laboratories, Inc.; polymethoxy bicyclic oxazolidine, available under the trade name Nuosept® C from Hills America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation. Suitable levels of preservative can range from about % to about 0.5%, alternatively from about 0.0002% to about 0.2%, alternatively from about 0.0003% to about 0.1%, by weight of the composition.

Suitable carriers for deodorant or antiperspirant products can include, water, alcohol, or combinations thereof. Useful alcohols include C₁-C₃ alcohols. In some aspects, the alcohol is ethanol.

Some examples of propellants suitable for deodorant or antiperspirant products include compressed air, nitrogen, inert gases, carbon dioxide, and mixtures thereof. Propellants may also include gaseous hydrocarbons like propane, n-butane, isobutene, cyclopropane, and mixtures thereof; e.g. A-46 (a mixture of isobutane, butane and propane), A-31 (isobutane), A-17 (n-butane), A-108 (propane), AP70 (a mixture of propane, isobutane and nbutane), AP40 (a mixture of propane, isobutene and n-butane), AP30 (a mixture of propane, isobutane and n-butane). Some non-limiting examples of propellants include 1,1-difluoroethane, 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof.

The term “primary structurant” as used herein means any material known or otherwise effective in providing suspending, gelling, viscosifying, solidifying, and/or thickening properties to the composition or which otherwise provide structure to the final product form. These primary structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof. Non-limiting examples of suitable primary structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fischer-Tropsch waxes, and microcrystalline wax; polyethylenes with molecular weight of 200 to 1000 daltons; solid triglycerides; behenyl alcohol, or combinations thereof.

Antiperspirant actives may include astringent metallic salts, especially inorganic and organic salts of aluminum, zirconium and zinc, as well as mixtures thereof. Even more specifically, antiperspirant actives may be selected from the group consisting of aluminum chloride, aluminum chlorohydrate, aluminum chlorohydrex, aluminum chlorohydrex PG, aluminum chlorohydrex PEG, aluminum dichlorohydrate, aluminum dichlorohydrex PG, aluminum dichlorohydrex PEG, aluminum sesquichlorohydrate, aluminum sesquichlorohydrex PG, aluminum sesquichlorohydrex PEG, aluminum sulfate, aluminum zirconium octachlorohydrate, aluminum zirconium octachlorohydrex GLY, aluminum zirconium pentachlorohydrate, aluminum zirconium pentachlorohydrex GLY, aluminum zirconium tetrachlorohydrate, aluminum zirconium trichlorohydrate, aluminum zirconium tetrachlorohydrate GLY and aluminum zirconium trichlorohydrate GLY.

Chassis ingredients suitable for deodorant or antiperspirant products may be an additional structurant such as stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; and solid triglycerides; behenyl alcohol, or combinations thereof; nonvolatile organic fluids such as mineral oil, PPG-14 butyl ether, isopropyl myristate, petrolatum, butyl stearate, cetyl octanoate, butyl myristate, myristyl myristate, C12-15 alkylbenzoate (e.g., Finsolv™), octyldodecanol, isostearyl isostearate, octododecyl benzoate, isostearyl lactate, isostearyl palmitate or isobutyl stearate; clay mineral powders such as talc, mica, sericite, silica, magnesium silicate, synthetic fluorphlogopite, calcium silicate, aluminum silicate, bentonite and montomorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titrate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, poly propylene, vinyl chloride polymer, tetrafluoroethylene polymer, boron nitride, fish scale guanine, laked tar color dyes, laked natural color dyes; and combinations thereof.

Volatile silicone solvents suitable for use in the antiperspirant compositions include, but are not limited to, solvent such as Cyclomethicone D-5; GE 7207 and GE 7158 (commercially available from General Electric Co.); Dow Corning 344; Dow Corning 345; Dow Corning 200; and DC1184 (commercially available from Dow Corning Corp.); and SWS-03314 (commercially available from SWS Silicones).

The gellant material may comprise saturated or unsaturated, substituted or unsubstituted, fatty alcohols or mixtures of fatty alcohols having from about 20 to about 60 carbons atoms, alternatively from about 20 to about 40 carbon atoms. In some embodiments, the gallant materials comprise combinations of the fatty alcohols. In some embodiments, the fatty alcohol gellants are may be saturated, unsubstituted monohydric alcohols or combinations thereof, which have a melting point of at less than about 110° C., alternatively from about 60° to about 110° C., alternatively between about 100° C. and 110° C. Specific examples of fatty alcohol gellants for use in the antiperspirant products that are commercially available include, but are not limited to, Unilin® 425, Unilin® 350, Unilin® 550 and Unilin® 700 (supplied by Petrolite).

A suitable buffering agent for deodorant or antiperspirant products may be alkaline, acidic or neutral. The buffer may be used in the composition or product for maintaining the desired pH. Suitable buffering agents include, for example, hydrochloric acid, sodium hydroxide, potassium hydroxide, and combinations thereof.

Non-limiting examples of suitable residue masking materials for use in the antiperspirant products include butyl stearate, diisopropyl adipate, petrolatum, nonvolatile silicones, octyldodecanol, phenyl trimethicone, isopropyl myristate, C₁₂₋₁₅ ethanol benzoates and PPG-14 Butyl Ether.

The deodorant or antiperspirant products disclosed herein may comprise other optional ingredients such as emulsifiers, distributing agents, antimicrobials, pharmaceutical or other topical actives, surfactants, and the like.

The nature, amount and type of ingredients does not warrant a more detailed description here, which in any case would not be exhaustive, the skilled person being able to select them on the basis of its general knowledge and according to intended form.

In some aspects, the composition comprises less than 95 wt % of water, relative to the total weight of the composition. In some aspects, the composition comprises less than 90 wt % of water, relative to the total weight of the composition. In some aspects, the composition comprises less than 85 wt % of water, relative to the total weight of the composition. In some aspects, the composition comprises less than 80 wt % of water, relative to the total weight of the composition. In some aspects, the composition comprises less than 75 wt % of water, relative to the total weight of the composition. In some aspects, the composition comprises less than 70 wt % of water, relative to the total weight of the composition. In some aspects, the composition comprises less than 65 wt % of water, relative to the total weight of the composition. In some aspects, the composition comprises less than 60 wt % of water, relative to the total weight of the composition. In some aspects, the composition comprises less than wt % of water, relative to the total weight of the composition. In some aspects, the composition comprises less than 50 wt %, or less than 40 wt %, or less than 30 wt %, or less than 20 wt %, or less than 10 wt % of water, relative to the total weight of the deodorant or antiperspirant composition. In some aspects, the composition is water-free.

Another object of the present invention is an antiperspirant or deodorant composition comprising:
a) at least one ingredient selected from the group consisting of (3E)-4-(2,6,6-trimethyl-1-cyclohexen-1-yl)-3-buten-2-one, (+−)-2-methylundecanal, (−)-(2E)-2-ethyl-4-[(1R)-2,2,3-trimethyl-3-cyclopenten-1-yl]-2-buten-1-ol, 1-(3,3/5,5-dimethyl-1-cyclohexen-1-yl)-4-penten-1-one, methyl 2,4-dihydroxy-3,6-dimethylbenzoate, (+)-(3R,5Z)-3-methyl-5-cyclopentadecen-1-one, patchouli oil, thymol, (+−)-(3E)-4-(2,6,6-trimethyl-1/2-cyclohexen-1-yl)-3-buten-2-one, 2-(3-phenylpropyl)pyridine, (+−)-(2E)-1-(2,6,6-trimethyl-2-cyclohexen-1-yl)-2-buten-1-one, (+−)-6/8-sec-butylquinoline, (+−)-3,5,6,6-tetramethyl-4-methylidene-2-heptanone, (4E)-3,4,5,6,6-pentamethyl-4-hepten-2-one, 3,4,5,6,6-pentamethyl-3-hepten-2-one, 1-(2-naphthyl)ethanone, methyl 2-((1RS,2RS)-3-oxo-2-pentylcyclopentyl)acetate, (2E,6Z)-2,6-nonadienal, (+−)-(E)-4-methyl-3-decen-5-ol and combinations thereof;
b) at least two ingredients selected from the group consisting of (3 aRS,5aSR,9aSR,9bRS)-3a,6,6,9a-tetramethyldodecahydronaphtho[2,1-b]furan, 4-methoxybenzaldehyde, 4-(4-hydroxyphenyl)-2-butanone, tricyclo[5.2.1.0(2,6)]dec-3/4-en-8-yl propanoate, 2-chromenone, (+−)-(3E)-3-methyl-4-(2,6,6-trimethyl-2-cyclohexen-1-yl)-3-buten-2-one, (+−)-(1E)-1-(2,6,6-trimethyl-2-cyclohexen-1-yl)-1-penten-3-one, (+−)-(2,5-dimethyl-2,3-dihydro-1H-inden-2-yl)methanol, (E)-2-ethoxy-undecanal, 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethanone, (+−)-2,6-dimethyl-7-octen-2-ol, 2-phenylethanol, (3Z)-3-hexen-1-ol, (4Z)-4-dodecenal, octanal, dec anal, 10-undecenal, 3-ethoxy-4-hydroxybenzaldehyde, 2-ethyl-3-hydroxy-4(4H)-pyranone, (E)-3,7-dimethyl-2,6-octadien-1-ol, (+−)-tetrahydro-2-isobutyl-4-methyl-4(2H)-pyranol, (1RS,2RS)-2,4-dimethyl-3-cyclohexene-1-carbaldehyde, (1RS,2SR)-2,4-dimethyl-3-cyclohexene-1-carb aldehyde, (+−)-3/4-(4-methyl-3-penten-1-yl)-3-cyclohexene-1-carbaldehyde and combinations thereof.

According to a particular embodiments, the antiperspirant or deodorant composition comprises:
a) at least two ingredients selected from the group consisting of (3E)-4-(2,6,6-trimethyl-1-cyclohexen-1-yl)-3-buten-2-one, (+−)-2-methylundecanal, (−)-(2E)-2-ethyl-4-[(1R)-2,2,3-trimethyl-3-cyclopenten-1-yl]-2-buten-1-ol, 1-(3,3/5,5-dimethyl-1-cyclohexen-1-yl)-4-penten-1-one, methyl 2,4-dihydroxy-3,6-dimethylbenzoate, (+)-(3R,5Z)-3-methyl-5-cyclopentadecen-1-one, patchouli oil, thymol, (+−)-(3E)-4-(2,6,6-trimethyl-1/2-cyclohexen-1-yl)-3-buten-2-one, 2-(3-phenylpropyl)pyridine, (+−)-(2E)-1-(2,6,6-trimethyl-2-cyclohexen-1-yl)-2-buten-1-one, (+−)-6/8-sec-butylquinoline, (+−)-3,5,6,6-tetramethyl-4-methylidene-2-heptanone, (4E)-3,4,5,6,6-pentamethyl-4-hepten-2-one, 3,4,5,6,6-pentamethyl-3-hepten-2-one, 1-(2-naphthyl)ethanone, methyl 2-((1RS,2RS)-3-oxo-2-pentylcyclopentyl)acetate, (2E,6Z)-2,6-nonadienal, (+−)-(E)-4-methyl-3-decen-5-ol and combinations thereof;
b) at least two ingredients selected from the group consisting of (3 aRS,5aSR,9aSR,9bRS)-3a,6,6,9a-tetramethyldodecahydronaphtho[2,1-b]furan, 4-methoxybenzaldehyde, 4-(4-hydroxyphenyl)-2-butanone, tricyclo[5.2.1.0(2,6)]dec-3/4-en-8-yl propanoate, 2-chromenone, (+−)-(3E)-3-methyl-4-(2,6,6-trimethyl-2-cyclohexen-1-yl)-3-buten-2-one, (+−)-(1E)-1-(2,6,6-trimethyl-2-cyclohexen-1-yl)-1-penten-3-one, (+−)-(2,5-dimethyl-2,3-dihydro-1H-inden-2-yl)methanol, (E)-2-ethoxy-undecanal, 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethanone, (+−)-2,6-dimethyl-7-octen-2-ol, 2-phenylethanol, (3Z)-3-hexen-1-ol, (4Z)-4-dodecenal, octanal, decanal, 10-undecenal, 3-ethoxy-4-hydroxybenzaldehyde, 2-ethyl-3-hydroxy-4(4H)-pyranone, (E)-3,7-dimethyl-2,6-octadien-1-ol, (+−)-tetrahydro-2-isobutyl-4-methyl-4(2H)-pyranol, (1RS,2RS)-2,4-dimethyl-3-cyclohexene-1-carbaldehyde, (1RS,2SR)-2,4-dimethyl-3-cyclohexene-1-carb aldehyde, (+−)-3/4-(4-methyl-3-penten-1-yl)-3-cyclohexene-1-carbaldehyde and combinations thereof.

According to a particular embodiments, the antiperspirant or deodorant composition comprises:
a) at least one ingredient selected from the group consisting of (3E)-4-(2,6,6-trimethyl-1-cyclohexen-1-yl)-3-buten-2-one, (+−)-2-methylundecanal, (−)-(2E)-2-ethyl-4-[(1R)-2,2,3-trimethyl-3-cyclopenten-1-yl]-2-buten-1-ol, 1-(3,3/5,5-dimethyl-1-cyclohexen-1-yl)-4-penten-1-one, methyl 2,4-dihydroxy-3,6-dimethylbenzoate, (+)-(3R,5Z)-3-methyl-5-cyclopentadecen-1-one, patchouli oil, thymol, (+−)-(3E)-4-(2,6,6-trimethyl-1/2-cyclohexen-1-yl)-3-buten-2-one, 2-(3-phenylpropyl)pyridine, (+−)-(2E)-1-(2,6,6-trimethyl-2-cyclohexen-1-yl)-2-buten-1-one, (+−)-6/8-sec-butylquinoline, (+−)-3,5,6,6-tetramethyl-4-methylidene-2-heptanone, (4E)-3,4,5,6,6-pentamethyl-4-hepten-2-one, 3,4,5,6,6-pentamethyl-3-hepten-2-one, 1-(2-naphthyl)ethanone, methyl 2-((1RS,2RS)-3-oxo-2-pentylcyclopentyl)acetate, (2E,6Z)-2,6-nonadienal, (+−)-(E)-4-methyl-3-decen-5-ol and combinations thereof;
b) at least three, four or five ingredients selected from the group consisting of (3 aRS,5aSR,9aSR,9bRS)-3a,6,6,9a-tetramethyldodecahydronaphtho[2,1-b]furan, 4-methoxybenzaldehyde, 4-(4-hydroxyphenyl)-2-butanone, tricyclo[5.2.1.0(2,6)]dec-3/4-en-8-yl propanoate, 2-chromenone, (+−)-(3E)-3-methyl-4-(2,6,6-trimethyl-2-cyclohexen-1-yl)-3-buten-2-one, (+−)-(1E)-1-(2,6,6-trimethyl-2-cyclohexen-1-yl)-1-penten-3-one, (+−)-(2,5-dimethyl-2,3-dihydro-1H-inden-2-yl)methanol, (E)-2-ethoxy-5-(1-propenyl)phenol, undecanal, 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethanone, (+−)-2,6-dimethyl-7-octen-2-ol, 2-phenylethanol, (3Z)-3-hexen-1-ol, (4Z)-4-dodecenal, octanal, decanal, 10-undecenal, 3-ethoxy-4-hydroxybenzaldehyde, 2-ethyl-3-hydroxy-4(4H)-pyranone, (E)-3,7-dimethyl-2,6-octadien-1-ol, (+−)-tetrahydro-2-isobutyl-4-methyl-4(2H)-pyranol, (1RS,2RS)-2,4-dimethyl-3-cyclohexene-1-carbaldehyde, (1RS,2SR)-2,4-dimethyl-3-cyclohexene-1-carb aldehyde, (+−)-3/4-(4-methyl-3-penten-1-yl)-3-cyclohexene-1-carbaldehyde and combinations thereof.

According to any embodiments of the invention, the antiperspirant or deodorant composition may comprise (3E)-4-(2,6,6-trimethyl-1-cyclohexen-1-yl)-3-buten-2-one in an amount of at least 4 wt. %, preferably of at least 6 wt. %, even more preferably of at least 10 wt. %, based on the total weight of the antiperspirant or deodorant composition. Particularly, the antiperspirant or deodorant composition may comprise (3E)-4-(2,6,6-trimethyl-1-cyclohexen-1-yl)-3-buten-2-one in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, even more preferably from 6 wt. % to 25 wt. % based on the total weight of the antiperspirant or deodorant composition.

According to any embodiments of the invention, the antiperspirant or deodorant composition may comprise (+−)-2-methylundecanal in an amount of at least 1.5 wt. %, preferably of at least 2 wt. %, even more preferably of at least 5 wt. %, based on the total weight of the antiperspirant or deodorant composition. Particularly, the antiperspirant or deodorant composition may comprise (+−)-2-methylundecanal in an amount of from 1.5 wt. % to 95 wt. %, preferably from 1.5 wt. % to 90 wt. %, preferably from 1.5 wt. % to 80 wt. %, preferably from 1.5 wt. % to 50 wt. %, preferably from 1.5 wt. % to 25 wt. %, preferably from 1.5 wt. % to wt. %, preferably from 1.5 wt. % to 5 wt. %, even more preferably from 2 wt. % to 5 wt. % based on the total weight of the antiperspirant or deodorant composition.

According to any embodiments of the invention, the antiperspirant or deodorant composition may comprise (−)-(2E)-2-ethyl-4-[(1R)-2,2,3-trimethyl-3-cyclopenten-1-yl]-2-buten-1-ol in an amount of at least 3 wt. %, preferably of at least 7 wt. %, even more preferably of at least wt. %, based on the total weight of the antiperspirant or deodorant composition. Particularly, the antiperspirant or deodorant composition may comprise (−)-(2E)-2-ethyl-4-[(1R)-2,2,3-trimethyl-3-cyclopenten-1-yl]-2-buten-1-ol in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 10 wt. %, even more preferably from 7 wt. % to 10 wt. % based on the total weight of the antiperspirant or deodorant composition.

According to any embodiments of the invention, the antiperspirant or deodorant composition may comprise 1-(3,3/5,5-dimethyl-1-cyclohexen-1-yl)-4-penten-1-one in an amount of at least 0.2 wt. %, preferably of at least 0.4 wt. %, even more preferably of at least 1 wt. %, based on the total weight of the antiperspirant or deodorant composition. Particularly, the antiperspirant or deodorant composition may comprise 1-(3,3/5,5-dimethyl-1-cyclohexen-1-yl)-4-penten-1-one in an amount of from 0.4 wt. % to 95 wt. %, preferably from 0.4 wt. % to wt. %, preferably from 0.4 wt. % to 80 wt. %, preferably from 0.4 wt. % to 50 wt. %, preferably from 0.4 wt. % to 25 wt. %, preferably from 0.4 wt. % to 10 wt. %, preferably from wt. % to 8 wt. %, even more preferably from 2 wt. % to 8 wt. % based on the total weight of the antiperspirant or deodorant composition.

According to any embodiments of the invention, the antiperspirant or deodorant composition may comprise methyl 2,4-dihydroxy-3,6-dimethylbenzoate in an amount of at least 1.5 wt. %, preferably of at least 2 wt. %, even more preferably of at least 5 wt. %, based on the total weight of the antiperspirant or deodorant composition. Particularly, the antiperspirant or deodorant composition may comprise methyl 2,4-dihydroxy-3,6-dimethylbenzoate in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 10 wt. %, preferably from 2 wt. % to 5 wt. %, even more preferably from 2 wt. % to 4 wt. % based on the total weight of the antiperspirant or deodorant composition.

According to any embodiments of the invention, the antiperspirant or deodorant composition may comprise (+)-(3R,5Z)-3-methyl-5-cyclopentadecen-1-one in an amount of at least 0.1 wt. %, preferably of at least 0.6 wt. %, even more preferably of at least 1 wt. %, based on the total weight of the antiperspirant or deodorant composition. Particularly, the antiperspirant or deodorant composition may comprise (+)-(3R,5Z)-3-methyl-5-cyclopentadecen-1-one in an amount of from 0.1 wt. % to 95 wt. %, preferably from 0.1 wt. % to 90 wt. %, preferably from 0.1 wt. % to 80 wt. %, preferably from 0.1 wt. % to 50 wt. %, preferably from 0.1 wt. % to 25 wt. %, preferably from 0.1 wt. % to 10 wt. %, preferably from 0.1 wt. % to 8 wt. %, even more preferably from 0.6 wt. % to 8 wt. % based on the total weight of the antiperspirant or deodorant composition.

According to any embodiments of the invention, the antiperspirant or deodorant composition may comprise patchouli oil in an amount of at least 1 wt. %, preferably of at least 3 wt. %, even more preferably of at least 10 wt. %, based on the total weight of the antiperspirant or deodorant composition. Particularly, the antiperspirant or deodorant composition may comprise patchouli oil in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 20 wt. %, preferably from 5 wt. % to 20 wt. %, even more preferably from 8 wt. % to 15 wt. % based on the total weight of the antiperspirant or deodorant composition.

According to any embodiments of the invention, the antiperspirant or deodorant composition may comprise thymol in an amount of at least 0.1 wt. %, preferably of at least 0.5 wt. %, even more preferably of at least 2 wt. %, based on the total weight of the antiperspirant or deodorant composition. Particularly, the antiperspirant or deodorant composition may comprise thymol in an amount of from 1 wt. % to 95 wt. %, preferably from 1 wt. % to 90 wt. %, preferably from 1 wt. % to 80 wt. %, preferably from 1 wt. % to 50 wt. %, preferably from 1 wt. % to 25 wt. %, preferably from 1 wt. % to 20 wt. %, preferably from 1 wt. % to 10 wt. %, even more preferably from 1 wt. % to 5 wt. % based on the total weight of the antiperspirant or deodorant composition.

According to any embodiments of the invention, the antiperspirant or deodorant composition may comprise (+−)-(3E)-4-(2,6,6-trimethyl-1/2-cyclohexen-1-yl)-3-buten-2-one in an amount of at least 1 wt. %, preferably of at least 2 wt. %, even more preferably of at least 4 wt. %, based on the total weight of the antiperspirant or deodorant composition. Particularly, the antiperspirant or deodorant composition may comprise (+−)-(3E)-4-(2,6,6-trimethyl-1/2-cyclohexen-1-yl)-3-buten-2-one in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, preferably from 4 wt. % to 20 wt. %, even more preferably from 4 wt. % to 15 wt. % based on the total weight of the antiperspirant or deodorant composition.

According to any embodiments of the invention, the antiperspirant or deodorant composition may comprise 2-(3-phenylpropyl)pyridine in an amount of at least 0.0001 wt. %, preferably of at least 0.0005 wt. %, even more preferably of at least 0.0007 wt. %, based on the total weight of the antiperspirant or deodorant composition. Particularly, the antiperspirant or deodorant composition may comprise 2-(3-phenylpropyl)pyridine in an amount of from 0.0007 wt. % to wt. %, preferably from 0.0007 wt. % to 90 wt. %, preferably from 0.0007 wt. % to 80 wt. %, preferably from 0.0007 wt. % to 50 wt. %, preferably from 0.0007 wt. % to 25 wt. %, preferably from 0.0007 wt. % to 20 wt. %, preferably from 0.0007 wt. % to 10 wt. %, preferably from wt. % to 5 wt. %, preferably from 0.0007 wt. % to 2 wt. %, preferably from 0.0007 wt. % to 1 wt. %, preferably from 0.0007 wt. % to 0.5 wt. %, preferably from 0.007 wt. % to 0.5 wt. %, preferably from 0.07 wt. % to 0.5 wt. %, even more preferably from 0.1 wt. % to 0.5 wt. % based on the total weight of the antiperspirant or deodorant composition.

According to any embodiments of the invention, the antiperspirant or deodorant composition may comprise (+−)-(2E)-1-(2,6,6-trimethyl-2-cyclohexen-1-yl)-2-buten-1-one in an amount of at least 0.1 wt. %, preferably of at least 0.3 wt. %, even more preferably of at least 0.5 wt. %, based on the total weight of the antiperspirant or deodorant composition. Particularly, the antiperspirant or deodorant composition may comprise (+−)-(2E)-1-(2,6,6-trimethyl-2-cyclohexen-1-yl)-2-buten-1-one in an amount of from 0.3 wt. % to 95 wt. %, preferably from wt. % to 90 wt. %, preferably from 0.3 wt. % to 80 wt. %, preferably from 0.3 wt. % to 50 wt. %, preferably from 0.3 wt. % to 25 wt. %, preferably from 0.3 wt. % to 20 wt. %, preferably from 0.3 wt. % to 10 wt. %, preferably from 0.3 wt. % to 5 wt. %, preferably from 0.3 wt. % to 2 wt. %, even more preferably from 0.3 wt. % to 1 wt. % based on the total weight of the antiperspirant or deodorant composition.

According to any embodiments of the invention, the antiperspirant or deodorant composition may comprise (+−)-6/8-sec-butylquinoline in an amount of at least 0.1 wt. %, preferably of at least 0.2 wt. %, even more preferably of at least 0.5 wt. %, based on the total weight of the antiperspirant or deodorant composition. Particularly, the antiperspirant or deodorant composition may comprise (+−)-6/8-sec-butylquinoline in an amount of from 0.2 wt. % to 95 wt. %, preferably from 0.2 wt. % to 90 wt. %, preferably from 0.2 wt. % to 80 wt. %, preferably from 0.2 wt. % to 50 wt. %, preferably from 0.2 wt. % to 25 wt. %, preferably from 0.2 wt. % to wt. %, preferably from 0.2 wt. % to 10 wt. %, preferably from 0.2 wt. % to 5 wt. %, even more preferably from 0.5 wt. % to 3 wt. % based on the total weight of the antiperspirant or deodorant composition.

According to any embodiments of the invention, the antiperspirant or deodorant composition may comprise (+−)-3,5,6,6-tetramethyl-4-methylidene-2-heptanone, (4E)-3,4,5,6,6-pentamethyl-4-hepten-2-one and 3,4,5,6,6-pentamethyl-3-hepten-2-one in an amount of at least 1 wt. %, preferably of at least 5 wt. %, even more preferably of at least 6 wt. %, based on the total weight of the antiperspirant or deodorant composition. Particularly, the antiperspirant or deodorant composition may comprise (+−)-3,5,6,6-tetramethyl-4-methylidene-2-heptanone, (4E)-3,4,5,6,6-pentamethyl-4-hepten-2-one and 3,4,5,6,6-pentamethyl-3-hepten-2-one, in an amount of from 5 wt. % to 95 wt. %, preferably from 5 wt. % to 90 wt. %, preferably from 5 wt. % to 80 wt. %, preferably from 5 wt. % to 50 wt. %, preferably from 5 wt. % to 25 wt. %, preferably from 5 wt. % to 20 wt. %, preferably from 5 wt. % to 10 wt. %, even more preferably from 6 wt. % to 10 wt. % based on the total weight of the antiperspirant or deodorant composition.

According to any embodiments of the invention, the antiperspirant or deodorant composition may comprise 1-(2-naphthyl)ethanone in an amount of at least 1 wt. %, preferably of at least 2 wt. %, even more preferably of at least 5 wt. %, based on the total weight of the antiperspirant or deodorant composition. Particularly, the antiperspirant or deodorant composition may comprise 1-(2-naphthyl)ethanone in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, preferably from 2 wt. % to 10 wt. %, even more preferably from 3 wt. % to 8 wt. % based on the total weight of the antiperspirant or deodorant composition.

According to any embodiments of the invention, the antiperspirant or deodorant composition may comprise methyl 2-((1RS,2RS)-3-oxo-2-pentylcyclopentyl)acetate in an amount of at least 5 wt. %, preferably of at least 10 wt. %, even more preferably of at least 50 wt. %, based on the total weight of the antiperspirant or deodorant composition. Particularly, the antiperspirant or deodorant composition may comprise methyl 2-((1RS,2RS)-3-oxo-2-pentylcyclopentyl)acetate in an amount of from 10 wt. % to 95 wt. %, preferably from 10 wt. % to 90 wt. %, preferably from 10 wt. % to 80 wt. %, preferably from 20 wt. % to 80 wt. %, preferably from 40 wt. % to 80 wt. %, even more preferably from 50 wt. % to 80 wt. %, based on the total weight of the antiperspirant or deodorant composition.

According to any embodiments of the invention, the antiperspirant or deodorant composition may comprise (2E,6Z)-2,6-nonadienal in an amount of at least 0.001 wt. %, preferably of at least 0.004 wt. %, even more preferably of at least 0.04 wt. %, based on the total weight of the antiperspirant or deodorant composition. Particularly, the antiperspirant or deodorant composition may comprise (2E,6Z)-2,6-nonadienal in an amount of from 0.004 wt. % to 95 wt. %, preferably from 0.004 wt. % to 90 wt. %, preferably from 0.004 wt. % to 80 wt. %, preferably from 0.004 wt. % to 50 wt. %, preferably from 0.004 wt. % to 25 wt. %, preferably from 0.004 wt. % to 20 wt. %, preferably from 0.004 wt. % to 10 wt. %, preferably from 0.004 wt. % to 5 wt. %, preferably from 0.004 wt. % to 1 wt. %, preferably from 0.004 wt. % to 0.1 wt. %, even more preferably from 0.01 wt. % to 0.1 wt. %, based on the total weight of the antiperspirant or deodorant composition.

According to any embodiments of the invention, the antiperspirant or deodorant composition may comprise (+−)-(E)-4-methyl-3-decen-5-ol in an amount of at least 1 wt. %, preferably of at least 5 wt. %, even more preferably of at least 10 wt. %, based on the total weight of the antiperspirant or deodorant composition. Particularly, the antiperspirant or deodorant composition may comprise (+−)-(E)-4-methyl-3-decen-5-ol in an amount of from 5 wt. % to 95 wt. %, preferably from 5 wt. % to 90 wt. %, preferably from 5 wt. % to 80 wt. %, preferably from 5 wt. % to 50 wt. %, preferably from 5 wt. % to 25 wt. %, preferably from 5 wt. % to 20 wt. %, even more preferably from 5 wt. % to 10 wt. %, based on the total weight of the antiperspirant or deodorant composition.

According to any embodiments of the invention, the antiperspirant or deodorant composition may comprise (3 aRS,5aSR,9aSR,9bRS)-3a,6,6,9a-tetramethyldodecahydronaphtho[2,1-b]furan in an amount of at least 1 wt. %, preferably of at least 2 wt. %, even more preferably of at least 10 wt. %, based on the total weight of the antiperspirant or deodorant composition. Particularly, the antiperspirant or deodorant composition may comprise (3 aRS,5aSR,9aSR,9bRS)-3a,6,6,9a-tetramethyldodecahydronaphtho[2,1-b]furan in an amount of from 1 wt. % to 95 wt. %, preferably from 1 wt. % to 90 wt. %, preferably from 1 wt. % to 80 wt. %, preferably from 1 wt. % to 50 wt. %, preferably from 1 wt. % to 25 wt. %, preferably from 1 wt. % to 20 wt. %, preferably from 1 wt. % to 10 wt. %, preferably from 1 wt. % to 5 wt. %, even more preferably from 2 wt. % to 5 wt. %, based on the total weight of the antiperspirant or deodorant composition.

According to any embodiments of the invention, the antiperspirant or deodorant composition may comprise 4-methoxybenzaldehyde in an amount of at least 1 wt. %, preferably of at least 2 wt. %, even more preferably of at least 10 wt. %, based on the total weight of the antiperspirant or deodorant composition. Particularly, the antiperspirant or deodorant composition may comprise 4-methoxybenzaldehyde in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, even more preferably from 10 wt. % to 20 wt. %, based on the total weight of the antiperspirant or deodorant composition.

According to any embodiments of the invention, the antiperspirant or deodorant composition may comprise 4-(4-hydroxyphenyl)-2-butanone in an amount of at least 1 wt. %, preferably of at least 3 wt. %, even more preferably of at least 7 wt. %, based on the total weight of the antiperspirant or deodorant composition. Particularly, the antiperspirant or deodorant composition may comprise 4-(4-hydroxyphenyl)-2-butanone in an amount of from 3 wt. % to 95 wt. %, preferably from 3 wt. % to 90 wt. %, preferably from 3 wt. % to 80 wt. %, preferably preferably from 3 wt. % to 50 wt. %, preferably from 3 wt. % to 25 wt. %, preferably from 3 wt. % to 20 wt. %, preferably from 3 wt. % to 10 wt. %, even more preferably from 7 wt. % to wt. %, based on the total weight of the antiperspirant or deodorant composition.

According to any embodiments of the invention, the antiperspirant or deodorant composition may comprise tricyclo[5.2.1.0(2,6)]dec-3/4-en-8-yl propanoate in an amount of at least 1 wt. %, preferably of at least 3 wt. %, even more preferably of at least 10 wt. %, based on the total weight of the antiperspirant or deodorant composition. Particularly, the antiperspirant or deodorant composition may comprise tricyclo[5.2.1.0(2,6)]dec-3/4-en-8-yl propanoate in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 20 wt. %, even more preferably from 10 wt. % to 20 wt. %, based on the total weight of the antiperspirant or deodorant composition.

According to any embodiments of the invention, the antiperspirant or deodorant composition may comprise 2-chromenone in an amount of at least 1 wt. %, preferably of at least 5 wt. %, even more preferably of at least 9 wt. %, based on the total weight of the antiperspirant or deodorant composition. Particularly, the antiperspirant or deodorant composition may comprise 2-chromenone in an amount of from 9 wt. % to 95 wt. %, preferably from 9 wt. % to wt. %, preferably from 9 wt. % to 80 wt %, preferably from 9 wt. % to 50 wt. %, preferably from 9 wt. % to 25 wt. %, preferably from 9 wt. % to 20 wt. %, even more preferably from 9 wt. % to 15 wt. %, based on the total weight of the antiperspirant or deodorant composition.

According to any embodiments of the invention, the antiperspirant or deodorant composition may comprise (+−)-(3E)-3-methyl-4-(2,6,6-trimethyl-2-cyclohexen-1-yl)-3-buten-2-one and (+−)-(1E)-1-(2,6,6-trimethyl-2-cyclohexen-1-yl)-1-penten-3-one in an amount of at least 1 wt. %, preferably of at least 5 wt. %, even more preferably of at least 7 wt. %, based on the total weight of the antiperspirant or deodorant composition. Particularly, the antiperspirant or deodorant composition may comprise (+−)-(3E)-3-methyl-4-(2,6,6-trimethyl-2-cyclohexen-1-yl)-3-buten-2-one and (+−)-(1E)-1-(2,6,6-trimethyl-2-cyclohexen-1-yl)-1-penten-3-one in an amount of from 7 wt. % to 95 wt. %, preferably from 7 wt. % to 90 wt. %, preferably from 7 wt. % to 80 wt. %, preferably from 7 wt. % to 50 wt. %, even more preferably from 7 wt. % to wt. %, based on the total weight of the antiperspirant or deodorant composition.

According to any embodiments of the invention, the antiperspirant or deodorant composition may comprise (+−)-(2,5-dimethyl-2,3-dihydro-1H-inden-2-yl)methanol in an amount of at least 1 wt. %, preferably of at least 4 wt. %, even more preferably of at least 10 wt. %, based on the total weight of the antiperspirant or deodorant composition. Particularly, the antiperspirant or deodorant composition may comprise (+−)-(2,5-dimethyl-2,3-dihydro-1H-inden-2-yl)methanol in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, preferably from 4 wt. % to 20 wt. %, preferably from 4 wt. % to 15 wt. %, even more preferably from 4 wt. % to 10 wt. %, based on the total weight of the antiperspirant or deodorant composition.

According to any embodiments of the invention, the antiperspirant or deodorant composition may comprise (E)-2-ethoxy-5-(1-propenyl)phenol in an amount of at least 0.005 wt. %, preferably of at least 0.05 wt. %, even more preferably of at least 0.5 wt. %, based on the total weight of the antiperspirant or deodorant composition. Particularly, the antiperspirant or deodorant composition may comprise (E)-2-ethoxy-5-(1-propenyl)phenol in an amount of from 0.005 wt. % to 95 wt. %, preferably from 0.005 wt. % to 90 wt. %, preferably from 0.005 wt. % to 80 wt. %, preferably from 0.005 wt. % to 50 wt. %, preferably from 0.005 wt. % to 25 wt. %, preferably from 0.005 wt. % to 20 wt. %, preferably from 0.005 wt. % to 15 wt. %, preferably from 0.005 wt. % to 10 wt. %, preferably from 0.05 wt. % to 10 wt. %, preferably from 0.5 wt. % to 10 wt. %, even more preferably from 1 wt. % to 10 wt. %, based on the total weight of the antiperspirant or deodorant composition.

According to any embodiments of the invention, the antiperspirant or deodorant composition may comprise undecanal in an amount of at least 0.05 wt. %, preferably of at least 0.5 wt. %, preferably of at least 1 wt. %, even more preferably of at least 3 wt. %, based on the total weight of the antiperspirant or deodorant composition. Particularly, the antiperspirant or deodorant composition may comprise undecanal in an amount of from 0.05 wt. % to 95 wt. %, preferably from 0.05 wt. % to 90 wt. %, preferably from 0.05 wt. % to 80 wt. %, preferably from 0.05 wt. % to 50 wt. %, preferably from 0.05 wt. % to 25 wt. %, preferably from 0.05 wt. % to 20 wt. %, preferably from 0.05 wt. % to 15 wt. %, preferably from 0.05 wt. % to 10 wt. %, preferably from 0.5 wt. % to 10 wt. %, preferably from 1 wt. % to 10 wt. %, even more preferably from 3 wt. % to 10 wt. %, based on the total weight of the antiperspirant or deodorant composition.

According to any embodiments of the invention, the antiperspirant or deodorant composition may comprise 1-[(1RS,2RS)-1,2,8,8-tetramethyl-1,2,3,4,5,6,7,8-octahydro-2-naphthalenyl]ethenone, 14(2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone and 1-[(2RS,3RS,8aRS)-2,3,8,8-tetramethyl-1,2,3,4,6,7,8,8a-octahydro-2-naphthalenyl]ethanone in an amount of at least 1 wt. %, preferably of at least 5 wt. %, preferably of at least 8 wt. %, even more preferably of at least 10 wt. %, based on the total weight of the antiperspirant or deodorant composition. Particularly, the antiperspirant or deodorant composition may comprise 1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethanone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone and 1-[(2RS,3RS,8aRS)-2,3,8,8-tetramethyl-1,2,3,4,6,7,8,8a-octahydro-2-naphthalenyl]ethanone in an amount of from 8 wt. % to 95 wt. %, preferably from 8 wt. % to 90 wt. %, preferably from 8 wt. % to 80 wt. %, preferably from 8 wt. % to 50 wt. %, even more preferably from 8 wt. % to wt. %, based on the total weight of the antiperspirant or deodorant composition.

According to any embodiments of the invention, the antiperspirant or deodorant composition may comprise (+−)-2,6-dimethyl-7-octen-2-ol in an amount of at least 10 wt. %, preferably of at least 50 wt. %, even more preferably of at least 70 wt. %, based on the total weight of the antiperspirant or deodorant composition. Particularly, the antiperspirant or deodorant composition may comprise (+−)-2,6-dimethyl-7-octen-2-ol in an amount of from 10 wt. % to wt. %, preferably from 20 wt. % to 95 wt. %, preferably from 30 wt. % to 95 wt. %, preferably from 40 wt. % to 95 wt. %, preferably from 50 wt. % to 95 wt. %, preferably from 60 wt. % to 95 wt. %, even more preferably from 70 wt. % to 95 wt. %, based on the total weight of the antiperspirant or deodorant composition.

According to any embodiments of the invention, the antiperspirant or deodorant composition may comprise 2-phenylethanol in an amount of at least 3 wt. %, preferably of at least 10 wt. %, even more preferably of at least 20 wt. %, based on the total weight of the antiperspirant or deodorant composition. Particularly, the antiperspirant or deodorant composition may comprise 2-phenylethanol in an amount of from 3 wt. % to 95 wt. %, preferably from 3 wt. % to 90 wt. %, preferably from 3 wt. % to 80 wt. %, preferably from 3 wt. % to 50 wt. %, preferably from 10 wt. % to 50 wt. %, even more preferably from 20 wt. % to wt. %, based on the total weight of the antiperspirant or deodorant composition.

According to any embodiments of the invention, the antiperspirant or deodorant composition may comprise (3Z)-3-hexen-1-ol in an amount of at least 0.1 wt. %, preferably of at least 0.4 wt. %, even more preferably of at least 1 wt. %, based on the total weight of the antiperspirant or deodorant composition. Particularly, the antiperspirant or deodorant composition may comprise (3Z)-3-hexen-1-ol in an amount of from 0.4 wt. % to 95 wt. %, preferably from 0.4 wt. % to 90 wt. %, preferably from 0.4 wt. % to 80 wt. %, preferably from 0.4 wt. % to 50 wt. %, preferably from 0.4 wt. % to 25 wt. %, preferably from 0.4 wt. % to 20 wt. %, preferably from 0.4 wt. % to 10 wt. %, preferably from 1 wt. % to 10 wt %, even more preferably from 2 wt. % to 8 wt. % based on the total weight of the antiperspirant or deodorant composition.

According to any embodiments of the invention, the antiperspirant or deodorant composition may comprise (4Z)-4-dodecenal in an amount of at least 0.01 wt. %, preferably of at least 0.1 wt. %, even more preferably of at least 0.3 wt. %, based on the total weight of the antiperspirant or deodorant composition. Particularly, the antiperspirant or deodorant composition may comprise (4Z)-4-dodecenal in an amount of from 0.01 wt. % to 95 wt. %, preferably from 0.01 wt. % to 90 wt. %, preferably from 0.01 wt. % to 80 wt. %, preferably from 0.01 wt. % to 50 wt. %, preferably from 0.01 wt. % to 25 wt. %, preferably from 0.01 wt. % to 20 wt. %, preferably from 0.01 wt. % to 10 wt. %, preferably from 0.01 wt. % to 5 wt %, preferably from 0.01 wt. % to 1 wt. %, even more preferably from 0.1 wt. % to 1 wt. % based on the total weight of the antiperspirant or deodorant composition.

According to any embodiments of the invention, the antiperspirant or deodorant composition may comprise octanal in an amount of at least 1 wt. %, preferably of at least 3 wt. %, even more preferably of at least 4 wt. %, based on the total weight of the antiperspirant or deodorant composition. Particularly, the antiperspirant or deodorant composition may comprise octanal in an amount of from 3 wt. % to 95 wt. %, preferably from 3 wt. % to 90 wt. %, preferably from 3 wt. % to 80 wt. %, preferably from 3 wt. % to 50 wt. %, preferably from 3 wt. % to 25 wt. %, preferably from 3 wt. % to 20 wt. %, preferably from 3 wt. % to 15 wt. %, preferably from 3 wt. % to 10 wt. %, even more preferably from 4 wt. % to 10 wt. %, based on the total weight of the antiperspirant or deodorant composition.

According to any embodiments of the invention, the antiperspirant or deodorant composition may comprise decanal in an amount of at least 1 wt. %, preferably of at least 5 wt. %, even more preferably of at least 10 wt. %, based on the total weight of the antiperspirant or deodorant composition. Particularly, the antiperspirant or deodorant composition may comprise decanal in an amount of from 5 wt. % to 95 wt. %, preferably from 5 wt. % to 90 wt. %, preferably from 5 wt. % to 80 wt. %, preferably from 5 wt. % to 50 wt. %, preferably from 5 wt. % to 25 wt. %, preferably from 5 wt. % to 20 wt. %, even more preferably from 1 wt. % to 20 wt. %, based on the total weight of the antiperspirant or deodorant composition.

According to any embodiments of the invention, the antiperspirant or deodorant composition may comprise 10-undecenal in an amount of at least 0.1 wt. %, preferably of at least 0.4 wt. %, even more preferably of at least 1 wt. %, based on the total weight of the antiperspirant or deodorant composition. Particularly, the antiperspirant or deodorant composition may comprise 10-undecenal in an amount of from 0.4 wt. % to 95 wt. %, preferably from 0.4 wt. % to 90 wt. %, preferably from 0.4 wt. % to 80 wt. %, preferably from 0.4 wt. % to 50 wt. %, preferably from 0.4 wt. % to 25 wt. %, preferably from 0.4 wt. % to 20 wt. %, preferably from 0.4 wt. % to 10 wt. %, preferably from 0.4 wt. % to 5 wt %, even more preferably from 1 wt. % to 5 wt. % based on the total weight of the antiperspirant or deodorant composition.

According to any embodiments of the invention, the antiperspirant or deodorant composition may comprise 3-ethoxy-4-hydroxybenzaldehyde in an amount of at least 1 wt. %, preferably of at least 2 wt. %, even more preferably of at least 10 wt. %, based on the total weight of the antiperspirant or deodorant composition. Particularly, the antiperspirant or deodorant composition may comprise 3-ethoxy-4-hydroxybenzaldehyde in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, preferably from 5 wt. % to 20 wt. %, even more preferably from 10 wt. % to 20 wt. %, based on the total weight of the antiperspirant or deodorant composition.

According to any embodiments of the invention, the antiperspirant or deodorant composition may comprise 2-ethyl-3-hydroxy-4(4H)-pyranone in an amount of at least 1 wt. %, preferably of at least 6 wt. %, even more preferably of at least 10 wt. %, based on the total weight of the antiperspirant or deodorant composition. Particularly, the antiperspirant or deodorant composition may comprise 2-ethyl-3-hydroxy-4(4H)-pyranone in an amount of from 1 wt. % to 95 wt. %, preferably from 1 wt. % to 90 wt. %, preferably from 1 wt. % to 80 wt. %, preferably from 1 wt. % to 50 wt. %, preferably from 1 wt. % to 25 wt. %, preferably from 1 wt. % to 20 wt. %, preferably from 1 wt. % to 10 wt. %, even more preferably from 6 wt. % to wt. %, based on the total weight of the antiperspirant or deodorant composition.

According to any embodiments of the invention, the antiperspirant or deodorant composition may comprise (E)-3,7-dimethyl-2,6-octadien-1-ol in an amount of at least 1 wt. %, preferably of at least 4 wt. %, even more preferably of at least 10 wt. %, based on the total weight of the antiperspirant or deodorant composition. Particularly, the antiperspirant or deodorant composition may comprise (E)-3,7-dimethyl-2,6-octadien-1-ol in an amount of from 4 wt. % to 95 wt. %, preferably from 4 wt. % to 90 wt. %, preferably from 4 wt. % to 80 wt. %, preferably from 4 wt. % to 50 wt. %, preferably from 4 wt. % to 25 wt. %, preferably from 4 wt. % to 20 wt. %, even more preferably from 10 wt. % to 20 wt. %, based on the total weight of the antiperspirant or deodorant composition.

According to any embodiments of the invention, the antiperspirant or deodorant composition may comprise (+−)-tetrahydro-2-isobutyl-4-methyl-4(2H)-pyranol in an amount of at least 1 wt. %, preferably of at least 9 wt. %, even more preferably of at least 20 wt. %, based on the total weight of the antiperspirant or deodorant composition. Particularly, the antiperspirant or deodorant composition may comprise (+−)-tetrahydro-2-isobutyl-4-methyl-4(2H)-pyranol in an amount of from 9 wt. % to 95 wt. %, preferably from 9 wt. % to 90 wt. %, preferably from 9 wt. % to 80 wt. %, preferably from 9 wt. % to 50 wt. %, preferably from 15 wt. % to 50 wt. %, even more preferably from 20 wt. % to 50 wt. %, based on the total weight of the antiperspirant or deodorant composition.

According to any embodiments of the invention, the antiperspirant or deodorant composition may comprise (1RS,2RS)-2,4-dimethyl-3-cyclohexene-1-carbaldehyde and (1RS,2SR)-2,4-dimethyl-3-cyclohexene-1-carbaldehyde in an amount of at least 1 wt. %, preferably of at least 2 wt. %, even more preferably of at least 10 wt. %, based on the total weight of the antiperspirant or deodorant composition. Particularly, the antiperspirant or deodorant composition may comprise (1RS,2RS)-2,4-dimethyl-3-cyclohexene-1-carbaldehyde and (1RS,2SR)-2,4-dimethyl-3-cyclohexene-1-carbaldehyde in an amount of from 2 wt. % to 95 wt. %, preferably from 2 wt. % to 90 wt. %, preferably from 2 wt. % to 80 wt. %, preferably from 2 wt. % to 50 wt. %, preferably from 2 wt. % to 25 wt. %, preferably from 2 wt. % to 20 wt. %, even more preferably from 2 wt. % to 10 wt. %, based on the total weight of the antiperspirant or deodorant composition.

According to any embodiments of the invention, the antiperspirant or deodorant composition may comprise (+−)-3/4-(4-methyl-3-penten-1-yl)-3-cyclohexene-1-carbaldehyde in an amount of at least 0.01 wt. %, preferably of at least 0.1 wt. %, preferably of at least 0.5 wt. %, even more preferably of at least 5 wt. %, based on the total weight of the antiperspirant or deodorant composition. Particularly, the antiperspirant or deodorant composition may comprise (+−)-3/4-(4-methyl-3-penten-1-yl)-3-cyclohexene-1-carbaldehyde in an amount of from 0.5 wt. % to 95 wt. %, preferably from 0.5 wt. % to 90 wt. %, preferably from 0.5 wt. % to 80 wt. %, preferably from 0.5 wt. % to 50 wt. %, preferably from 0.5 wt. % to 25 wt. %, preferably from 0.5 wt. % to 20 wt. %, preferably from 0.5 wt. % to 10 wt. %, even more preferably from 1 wt. % to 10 wt. %, based on the total weight of the antiperspirant or deodorant composition.

In some aspects, the antiperspirant or deodorant composition may be added to a perfuming composition comprising a perfumery carrier and a perfumery base; and optionally at least one perfumery adjuvant wherein perfumery carrier, perfumery base and perfumery adjuvant have the same meaning as defined above. So another object of the invention is a perfuming composition comprising
a. an antiperspirant or deodorant composition as defined above;
b. at least one ingredient selected from the group consisting of a perfumery carrier as defined above and a perfumery base as defined above; and
c. optionally at least one perfumery adjuvant as defined above.

In some aspects, the antiperspirant or deodorant composition or the perfuming composition comprising the antiperspirant or deodorant composition may be added to an antiperspirant or deodorant product comprising at least one antiperspirant or deodorant active having the same meaning as defined above. So another object of the invention is a antiperspirant or deodorant product comprising
a. an antiperspirant or deodorant composition as defined above or the perfuming composition as defined above; and
b. at least one antiperspirant or deodorant active having the same definition as defined above.

The present invention is best illustrated but is not limited to the following examples.

### EXAMPLES

### Example 1: Determining Performance and Effective Concentrations of Malodor Reducing Agents for the Reduction of the Perception of Sweat Body Odor by an Expert Evaluator

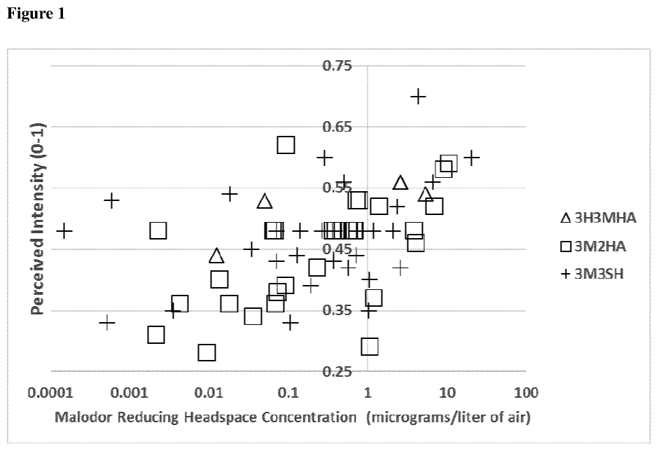

The effectiveness of perfuming materials in compositions to reduce the perception of, for example, axillary sweat malodors, was evaluated by an expert perfumer by 1) selecting target axillary malodor components, 2) delivering target malodor components and candidate malodor reducing materials to the nose of an expert perfumer at carefully controlled concentrations, 3) having the expert perfumer identify the concentration at which malodor reduction was obtained, and 4) rating the degree of reduction achieved at the concentration identified in step 3. Table 1 shows candidate malodor reducing materials that showed effectiveness when tested according to the this method against 3 components of axillary sweat malodor; 3-methyl-3-hydroxyhexanoic acid, 3-methyl-2-hexenoic acid, and 3-methyl-3-sulphanylhexanol. Two surprising results were be observed. The first is that not all tested materials yielded complete malodor reduction, often differing depending on which component they were tested against. The second is that the concentrations required for malodor reduction differed by several orders of magnitude across the tested materials and showed no correlation with the intensity they delivered at those identified concentrations (FIG. 1). Intensity and concentration were material-dependent and spanned a wide range of values on both axes. In some cases materials were required to be at significantly higher intensity than that of the target malodor, and in others, significantly lower. Thus, neither the required concentrations, nor performance ratings could be predicted a priori by one skilled in the art, yet are necessary and sufficient for designing malodor reducing compositions for use in perfuming products.

In this method, a dynamic multi-channel olfactometer was used to precisely deliver known gas phase concentrations of the test materials, using forced evaporation, into a stream of air emanating from a smelling port. The appropriate concentration of the malodor component was established prior to its being mixed with the candidate malodor reducing substance. The concentration was set at a concentration sufficient to produce an intensity of 0.5 (mid-intensity) on a scale of 0-1. The expert perfumer then dynamically adjusted the concentration of the candidate malodor reducing substance to determine the effective concentration, while maintaining a fixed concentration of the malodor component. Once the appropriate concentration had been determined, the expert perfumer evaluated the perception of the mixtures repeatedly over a period of several minutes, taking breaks in between sniffs to avoid adaptation to the components being tested, before providing a rating.

Table 1 shows the IUPAC name of the candidate malodor reducing material, the abbreviated name of the axillary sweat malodor component it was evaluated against, the headspace concentration identified as providing an effective level and the expert rating of the degree of malodor reduction at the effective level. Tests were performed in a forced-evaporation, dynamic olfactometer, with malodor component concentrations held fixed at a mid-intensity. Ratings of effectiveness were based in the following scale: −2 (significant increase in malodor), −1 (slight increase in malodor), 0 (no effect), 1 (slight malodor reduction), 2 (good malodor reduction), 3 (complete malodor reduction). The malodor (MO) component abbreviations are 3H3MHA (3-methyl-3-hydroxyhexanoic acid), 3M2HA (3-methyl-2-hexenoic acid), 3M3SH (3-methyl-3-sulphanylhexanol).

TABLE 1

| | | | EFFECTIVE |
|---|---|---|---|
| | | EXPERT | CONCENTRATION |
| IUPAC NAME | MO | RATING | (micrograms/L AIR) |
| 3-PHENYLPROPANAL | 3M2HA | 3 | 2.60E+00 |
| 3-PHENYLPROPANAL | 3M3SH | 3 | 2.60E+00 |
| 2-METHOXYNAPHTHALENE | 3M2HA | 3 | 1.25E−02 |
| 2-METHOXYNAPHTHALENE | 3M3SH | 3 | 5.00E−02 |
| (+−)-2,6-DIMETHYL-2-OCTANOL | 3M2HA | 2 | 5.40E+00 |
| (+−)-2,6-DIMETHYL-2-OCTANOL | 3M3SH | 2 | 1.08E+01 |
| 2-ETHYL-3-HYDROXY-4(4H)-PYRANONE | 3M2HA | 3 | 1.83E−02 |
| 2-ETHYL-3-HYDROXY-4(4H)-PYRANONE | 3M3SH | 2 | 1.83E−02 |
| (+−)-2-(4-METHYL-3-CYCLOHEXEN-1-YL)- | 3M2HA | 3 | 1.08E+00 |
| 2-PROPANYL ACETATE | | | |
| (+−)-2-(4-METHYL-3-CYCLOHEXEN-1-YL)- | 3M3SH | 3 | 1.08E+00 |
| 2-PROPANYL ACETATE | | | |
| ALLYL HEPTANOATE | 3M2HA | 3 | 4.13E+00 |
| (E)-3,7-DIMETHYL-2,6-OCTADIEN-1-OL | 3M2HA | 3 | 7.00E−01 |
| (E)-3,7-DIMETHYL-2,6-OCTADIEN-1-OL | 3M3SH | 3 | 1.40E+00 |
| ETHYL 2-METHYL-1,3-DIOXOLANE-2-ACETATE | 3M2HA | 2 | 7.07E+00 |
| (2E)-3-PHENYL-2-PROPEN-1-OL | 3M3SH | 2 | 6.50E−02 |
| 3-(2/4-ETHYLPHENYL)-2,2-DIMETHYLPROPANAL | 3M2HA | 3 | 3.67E−01 |
| 3-(2/4-ETHYLPHENYL)-2,2-DIMETHYLPROPANAL | 3M3SH | 2 | 7.33E−01 |
| (+−)-2,2,5-TRIMETHYL-5-PENTYLCYCLOPENTANONE | 3M2HA | 2 | 1.22E+00 |
| (+−)-3,7-DIMETHYL-3-OCTANOL | 3M2HA | 2 | 9.27E+00 |
| (2RS,4SR)-4-METHYL-2-(2-METHYL-1-PROPEN-1- | 3M2HA | 2 | 6.83E−01 |
| YL)TETRAHYDRO-2H-PYRAN | | | |
| (2RS,4SR)-4-METHYL-2-(2-METHYL-1-PROPEN-1- | 3M3SH | 1 | 6.83E−01 |
| YL)TETRAHYDRO-2H-PYRAN | | | |
| METHYL 2-OCTYNOATE | 3M2HA | 3 | 4.67E−01 |
| METHYL 2-OCTYNOATE | 3M3SH | 3 | 4.67E−01 |
| (+−)-2-METHYLUNDECANAL | 3M2HA | 2 | 2.33E−03 |
| (3aRS,5aSR,9aSR,9bRS)-3a,6,6,9a- | 3M2HA | 3 | 4.33E−03 |
| TETRAMETHYLDODECAHYDRONAPHTHO[2,1-b]FURAN | | | |
| (3aRS,5aSR,9aSR,9bRS)-3a,6,6,9a- | 3M3SH | 3 | 2.17E−03 |
| [symbol] ETRAMETHYLDODECAHYDRONAPHTHO[2,1-b]FURAN | | | |
| (2,2-DIMETHOXYETHYL)BENZENE | 3M2HA | 3 | 3.92E+00 |
| (2,2-DIMETHOXYETHYL)BENZENE | 3M3SH | 2 | 3.92E+00 |
| (+−)-METHYL 2,2-DIMETHYL-6- | 3H3MHA | 3 | 8.00E−01 |
| METHYLIDENECYCLOHEXANECARBOXYLATE | | | |
| (+−)-METHYL 2,2-DIMETHYL-6- | 3M2HA | 3 | 4.00E−01 |
| METHYLIDENECYCLOHEXANECARBOXYLATE | | | |
| (+−)-METHYL 2,2-DIMETHYL-6- | 3M3SH | 2 | 4.00E−01 |
| METHYLIDENECYCLOHEXANECARBOXYLATE | | | |
| ETHYL (E)-3-PHENYL-2-PROPENOATE | 3M2HA | 2 | 7.33E−02 |
| ETHYL (E)-3-PHENYL-2-PROPENOATE | 3M3SH | 2 | 3.67E−02 |
| METHYL (E)-3-PHENYL-2-PROPENOATE | 3M2HA | 3 | 7.00E−02 |
| METHYL (E)-3-PHENYL-2-PROPENOATE | 3M3SH | 2 | 9.33E−02 |
| (+−)-(5E)-3-METHYL-5-CYCLOPENTADECEN-1-ONE | 3M2HA | 2 | 2.33E−01 |
| (+−)-(5E)-3-METHYL-5-CYCLOPENTADECEN-1-ONE | 3M3SH | 1 | 5.83E−01 |
| 4-METHOXYBENZALDEHYDE | 3M2HA | 2 | 6.83E−02 |
| 10-UNDECENAL | 3M2HA | 3 | 9.50E−03 |
| 10-UNDECENAL | 3M3SH | 2 | 9.50E−02 |
| 1-(2-NAPHTHYL)ETHANONE | 3H3MHA | 3 | 1.37E−02 |
| 1-(2-NAPHTHYL)ETHANONE | 3M2HA | 3 | 1.37E−02 |
| 1-(2-NAPHTHYL)ETHANONE | 3M3SH | 2 | 3.42E−02 |
| 4-PHENYL-2-BUTANONE | 3M2HA | 3 | 7.50E−01 |
| 4-PHENYL-2-BUTANONE | 3M3SH | 2 | 3.75E−01 |
| (10E)-OXACYCLOHEPTADEC-10-EN-2-ONE | 3M2HA | 2 | 7.17E−02 |
| (10E)-OXACYCLOHEPTADEC-10-EN-2-ONE | 3M3SH | 2 | 2.87E−01 |
| [symbol] +−)-(6E)-3,7-DIMETHYL-1,6-NONADIEN-3-YL ACETATE | 3M3SH | 2 | 6.67E+00 |
| 4-(4-HYDROXYPHENYL)-2-BUTANONE | 3M2HA | 3 | 1.50E−04 |
| 4-(4-HYDROXYPHENYL)-2-BUTANONE | 3M3SH | 1 | 6.00E−04 |
| ETHYL 1RS,2SR,3SR,4SR-3- | 3H3MHA | 3 | 1.03E+00 |
| ISOPROPYLBICYCLO[2.2.1]HEPT-5-ENE-2- | | | |
| CARBOXYLATE | | | |
| ETHYL 1RS,2SR,3SR,4SR-3- | 3M2HA | 2 | 2.58E+00 |
| ISOPROPYLBICYCLO[2.2.1]HEPT-5-ENE-2- | | | |
| CARBOXYLATE | | | |
| ETHYL 1RS,2SR,3SR,4SR-3- | 3M3SH | 3 | 2.07E+01 |
| ISOPROPYLBICYCLO[2.2.1]HEPT-5-ENE-2- | | | |
| CARBOXYLATE | | | |
| [symbol] HYL TRICYCLO[5.2.1.0.(2,6)]DECANE-2-CARBOXYLATE | 3M2HA | 3 | 1.07E−01 |
| 4-METHOXYBENZYL ACETATE | 3M2HA | 3 | 1.18E+00 |
| 4-METHOXYBENZYL ACETATE | 3M3SH | 2 | 2.37E+00 |
| 3-ETHOXY-4-HYDROXYBENZALDEHYDE | 3M2HA | 3 | 5.17E−04 |
| (3Z)-3-HEXEN-1-OL | 3M2HA | 3 | 1.92E−01 |
| (3Z)-3-HEXEN-1-OL | 3M3SH | 3 | 3.83E−01 |
| METHYL 2-((1RS,2RS)-3-OXO-2- | 3H3MHA | 3 | 5.73E−01 |
| PENTYLCYCLOPENTYL)ACETATE | | | |
| METHYL 2-((1RS,2RS)-3-OXO-2- | 3M2HA | 2 | 7.17E−01 |
| PENTYLCYCLOPENTYL)ACETATE | | | |
| METHYL 2-((1RS,2RS)-3-OXO-2- | 3M3SH | 2 | 7.17E−01 |
| PENTYLCYCLOPENTYL)ACETATE | | | |
| (+−)-2,6-DIMETHYL-7-OCTEN-2-OL | 3M2HA | 2 | 4.33E+00 |
| (+−)-6-SEC-BUTYLQUINOLINE | 3M3SH | 3 | 1.83E−02 |
| PENTYL 2-HYDROXYBENZOATE | 3M3SH | 3 | 5.00E−01 |
| ALLYL HEXANOATE | 3M2HA | 3 | 1.06E+00 |
| ALLYL HEXANOATE | 3M3SH | 3 | 2.12E+00 |
| (+−)-3,7-DIMETHYL-6-OCTENAL | 3H3MHA | 3 | 1.30E−01 |
| (+−)-3,7-DIMETHYL-6-OCTENAL | 3M2HA | 2 | 1.30E−01 |
| METHYL 2,4-DIHYDROXY-3,6-DIMETHYLBENZOATE | 3M2HA | 3 | 3.50E−03 |
| METHYL 2,4-DIHYDROXY-3,6-DIMETHYLBENZOATE | 3M3SH | 3 | 3.50E−03 |
| TRICYCLO[5.2.1.0(2,6)]DEC-3/4-EN-8-YL PROPANOATE | 3M2HA | 3 | 7.08E−02 |
| TRICYCLO[5.2.1.0(2,6)]DEC-3/4-EN-8-YL PROPANOATE | 3M3SH | 2 | 1.42E−01 |
| (2RS,4SR)-4-METHYL-2-PHENYLTETRAHYDRO-2H-PYRAN | 3M3SH | 3 | 2.67E−01 |
| (3E)-4-(2,6,6-TRIMETHYL-1-CYCLOHEXEN-1-YL)-3- | 3M2HA | 3 | 5.83E−02 |
| BUTEN-2-ONE | | | |
| (3E)-4-(2,6,6-TRIMETHYL-1-CYCLOHEXEN-1-YL)-3- | 3M3SH | 3 | 1.17E−01 |
| BUTEN-2-ONE | | | |
| [symbol] ,3/5,5-DIMETHYL-1-CYCLOHEXEN-1-YL)-4-PENTEN-1- | 3H3MHA | 3 | 1.22E+00 |
| ONE | | | |
| [symbol] ,3/5,5-DIMETHYL-1-CYCLOHEXEN-1-YL)-4-PENTEN-1- | 3M2HA | 3 | 2.53E−03 |
| ONE | | | |
| [symbol] ,3/5,5-DIMETHYL-1-CYCLOHEXEN-1-YL)-4-PENTEN-1- | 3M3SH | 3 | 1.22E+00 |
| ONE | | | |
| (+)-(3R,5Z)-3-METHYL-5-CYCLOPENTADECEN-1-ONE | 3H3MHA | 2 | 5.50E−01 |
| (+)-(3R,5Z)-3-METHYL-5-CYCLOPENTADECEN-1-ONE | 3M2HA | 2 | 1.65E−01 |
| (+)-(3R,5Z)-3-METHYL-5-CYCLOPENTADECEN-1-ONE | 3M3SH | 2 | 2.75E−01 |
| PATCHOULI BIO | 3H3MHA | 3 | 4.00E−02 |
| PATCHOULI BIO | 3M2HA | 3 | 4.00E−02 |
| PATCHOULI BIO | 3M3SH | 3 | 1.20E−01 |
| THYMOL | 3M2HA | 3 | 5.17E−02 |
| THYMOL | 3M3SH | 3 | 1.03E−01 |
| [symbol] −)-(3E)-4-(2,6,6-TRIMETHYL-1/2-CYCLOHEXEN-1-YL)-3- | 3H3MHA | 3 | 2.00E−01 |
| BUTEN-2-ONE | | | |
| [symbol] −)-(3E)-4-(2,6,6-TRIMETHYL-1/2-CYCLOHEXEN-1-YL)-3- | 3M2HA | 3 | 4.00E−01 |
| BUTEN-2-ONE | | | |
| [symbol] −)-(3E)-4-(2,6,6-TRIMETHYL-1/2-CYCLOHEXEN-1-YL)-3- | 3M3SH | 2 | 8.00E−01 |
| BUTEN-2-ONE | | | |
| 2-(3-PHENYLPROPYL)PYRIDINE | 3H3MHA | 3 | 1.43E−04 |
| 2-(3-PHENYLPROPYL)PYRIDINE | 3M2HA | 3 | 1.43E−04 |
| (+−)-(2E)-1-(2,6,6-TRIMETHYL-2-CYCLOHEXEN-1-YL)-2- | 3H3MHA | 3 | 1.08E−02 |
| BUTEN-1-ONE | | | |
| (+−)-(2E)-1-(2,6,6-TRIMETHYL-2-CYCLOHEXEN-1-YL)-2- | 3M2HA | 2 | 1.30E−02 |
| BUTEN-1-ONE | | | |
| (+−)-3,5,6,6-TETRAMETHYL-4-METHYLIDENE-2- | | | |
| [symbol] PTANONE, (4E)-3,4,5,6,6-PENTAMETHYL-4-HEPTEN-2- | 3H3MHA | 3 | 2.19E+00 |
| ONE, 3,4,5,6,6-PENTAMETHYL-3-HEPTEN-2-ONE | | | |
| (+−)-3,5,6,6-TETRAMETHYL-4-METHYLIDENE-2- | 3M2HA | 3 | 8.77E+00 |
| [symbol] PTANONE, (4E)-3,4,5,6,6-PENTAMETHYL-4-HEPTEN-2- | | | |
| ONE, 3,4,5,6,6-PENTAMETHYL-3-HEPTEN-2-ONE | | | |
| (+−)-3,5,6,6-TETRAMETHYL-4-METHYLIDENE-2- | 3M3SH | 3 | 4.38E+00 |
| [symbol] PTANONE, (4E)-3,4,5,6,6-PENTAMETHYL-4-HEPTEN-2- | | | |
| ONE, 3,4,5,6,6-PENTAMETHYL-3-HEPTEN-2-ONE | | | |
| (2E,6Z)-2,6-NONADIENAL | 3H3MHA | 3 | 1.13E−03 |
| (2E,6Z)-2,6-NONADIENAL | 3M2HA | 3 | 2.83E−03 |
| (2E,6Z)-2,6-NONADIENAL | 3M3SH | 3 | 5.67E−04 |
| (+−)-(E)-4-METHYL-3-DECEN-5-OL | 3M2HA | 3 | 2.13E−01 |
| (+−)-(E)-4-METHYL-3-DECEN-5-OL | 3M3SH | 3 | 4.27E−01 |
| 2-CHROMENONE | 3M2HA | 3 | 9.83E−03 |
| 2-CHROMENONE | 3M3SH | 3 | 7.87E−02 |
| (+−)-(3E)-3-METHYL-4-(2,6,6-TRIMETHYL-2- | | | |
| [symbol] CYCLOHEXEN-1-YL)-3-BUTEN-2-ONE, (+−)-(1E)-1-(2,6,6- | 3M2HA | 3 | 1.08E−01 |
| TRIMETHYL-2-CYCLOHEXEN-1-YL)-1-PENTEN-3-ONE | | | |
| (+−)-(3E)-3-METHYL-4-(2,6,6-TRIMETHYL-2- | | | |
| [symbol] CYCLOHEXEN-1-YL)-3-BUTEN-2-ONE, (+−)-(1E)-1-(2,6,6- | 3M3SH | 1 | 2.17E−01 |
| TRIMETHYL-2-CYCLOHEXEN-1-YL)-1-PENTEN-3-ONE | | | |
| (+−)-(2,5-DIMETHYL-2,3-DIHYDRO-1H-INDEN-2- | 3H3MHA | 3 | 3.13E−02 |
| YL)METHANOL | | | |
| (+−)-(2,5-DIMETHYL-2,3-DIHYDRO-1H-INDEN-2- | 3M2HA | 3 | 4.70E−02 |
| YL)METHANOL | | | |
| (+−)-(2,5-DIMETHYL-2,3-DIHYDRO-1H-INDEN-2- | 3M3SH | 3 | 7.83E−02 |
| YL)METHANOL | | | |
| (E)-2-ETHOXY-5-(1-PROPENYL)PHENOL | 3H3MHA | 3 | 4.50E−03 |
| (E)-2-ETHOXY-5-(1-PROPENYL)PHENOL | 3M2HA | 3 | 1.80E−02 |
| (E)-2-ETHOXY-5-(1-PROPENYL)PHENOL | 3M3SH | 3 | 4.50E−02 |
| UNDECANAL | 3M2HA | 3 | 5.42E−02 |
| UNDECANAL | 3M3SH | 3 | 2.17E−01 |
| 1-[(1RS,2RS)-1,2,8,8-TETRAMETHYL-1,2,3,4,5,6,7,8- | 3M2HA | 3 | 3.33E−01 |
| OCTAHYDRO-2-NAPHTHALENYL]ETHENONE, 1- | | | |
| ((2RS,3RS)-2,3,8,8-TETRAMETHYL-1,2,3,4,5,6,7,8- | | | |
| OCTAHYDRONAPHTHALEN-2-YL)ETHANONE, 1- | | | |
| [(2RS,3RS,8ARS)-2,3,8,8-TETRAMETHYL-1,2,3,5,6,7,8,8A- | | | |
| OCTAHYDRO-2-NAPHTHALENYL]ETHENONE, 1- | | | |
| [(1RS,2RS,8ASR)-1,2,8,8-TETRAMETHYL-1,2,3,5,6,7,8,8A- | | | |
| OCTAHYDRO-2-NAPHTHALENYL]ETHENONE, 1- | | | |
| [(2RS,3RS,8ARS)-2,3,8,8-TETRAMETHYL-1,2,3,4,6,7,8,8A- | | | |
| OCTAHYDRO-2-NAPHTHALENYL]ETHANONE | | | |
| 1-[(1RS,2RS)-1,2,8,8-TETRAMETHYL-1,2,3,4,5,6,7,8- | 3M3SH | 2 | 3.33E−01 |
| OCTAHYDRO-2-NAPHTHALENYL]ETHENONE, 1- | | | |
| ((2RS,3RS)-2,3,8,8-TETRAMETHYL-1,2,3,4,5,6,7,8- | | | |
| OCTAHYDRONAPHTHALEN-2-YL)ETHANONE, 1- | | | |
| [(2RS,3RS,8ARS)-2,3,8,8-TETRAMETHYL-1,2,3,5,6,7,8,8A- | | | |
| OCTAHYDRO-2-NAPHTHALENYL]ETHENONE, 1- | | | |
| [(1RS,2RS,8ASR)-1,2,8,8-TETRAMETHYL-1,2,3,5,6,7,8,8A- | | | |
| OCTAHYDRO-2-NAPHTHALENYL]ETHENONE, 1- | | | |
| [(2RS,3RS,8ARS)-2,3,8,8-TETRAMETHYL-1,2,3,4,6,7,8,8A- | | | |
| OCTAHYDRO-2-NAPHTHALENYL]ETHANONE | | | |
| 2-PHENYLETHANOL | 3M2HA | 3 | 5.42E−01 |
| 2-PHENYLETHANOL | 3M3SH | 2 | 1.08E+00 |
| (4Z)-4-DODECENAL | 3M2HA | 2 | 1.03E−04 |
| (4Z)-4-DODECENAL | 3M3SH | 1 | 2.07E−04 |
| OCTANAL | 3M2HA | 3 | 6.17E−01 |
| OCTANAL | 3M3SH | 2 | 3.08E−01 |
| DECANAL | 3M2HA | 3 | 5.00E−01 |
| DECANAL | 3M3SH | 2 | 2.50E−01 |

[symbol]

indicates data missing or illegible when filed

### Example 2: Performance at Mid-Intensity Dosages of Malodor Reducing Agents for the Reduction of the Perception of Sweat Body Odor Components by Naïve Panelists

The performance of fragrancing products was evaluated by non-expert consumers under passive evaporation conditions. This was achieved by: 1) selecting target axillary malodor components, 2) delivering target malodor components and candidate malodor reducing materials (separately) to the noses of naïve panelists at a range of dosages established under passive evaporation conditions 3) having the panelists evaluate the overall intensity of each presented sample, 4) determining the average mid-intensity concentration for both the malodors and malodor reducing candidates of interest, 5) delivering samples containing a malodor component and a malodor reducing candidate of interest mixed together at the dosages determined in step 4, along with separate control samples containing either alone or neither, to the noses of panelists, 6) having panelists evaluate the malodor intensity for each presented sample, and 7) determining the degree of malodor reduction produced by the candidate malodor reducing material by comparing the malodor intensity ratings of the mixture to those of the control samples.

Table 2 shows a selection of candidate malodor reducing materials that showed a statistically significant malodor reduction as well as materials that did not when tested according to the method above against 4 components of axillary sweat malodor; 4-methyl octanoic acid, 3-methyl-3-hydroxyhexanoic acid, 3-methyl-2-hexenoic acid, and 3-methyl-3-sulphanylhexanol. For each condition tested, steps 2 and 5 of the above testing method were achieved by placing two small cellulose pads (1×1 inch) into a glass jar with a tight-fitting flapper lid; applying either 0.25 ml of DiPG or 0.25 ml of DiPG containing appropriately dosed malodor to one and 0.25 ml of DiPG or 0.25 ml of DiPG containing appropriately dosed candidate malodor reducing material; allowing the jar to equilibrate for 4 hours at standard room temperature and pressure; then presenting it to a panelist stationed in a sensory booth. The panelist was asked to open the lid and sniff for −15 seconds with their nose close to, but not inside, the opening of the jar and without breathing into the jar, to ensure optimal sampling of the jar headspace. Individual panelist scores were aggregated to derive the either the mean intensity at each dosage (for step 4), or the mean malodor intensity (for step 7). To determine whether the malodor intensity of the malodor and candidate malodor reducing material mixture was statistically different from the malodor alone, a Student's t-test was performed and results passing a significance level of 95% were assumed to be statistically significant (denoted by a * in Table 2).

Table 2 shows the IUPAC name of the candidate malodor reducing material, the abbreviated name of the axillary sweat malodor component it was evaluated against, the malodor dosage (diluted in DiPG and placed on a cellulose pad), the candidate malodor reducing material (“Test Compound”), the degree of malodor reduction produced by the test compound at the given dosages of MO and test compound, the significance of the reduction according to a Student's t-test with α=0.95, and the number (N) of panelists tested. Tests were performed in a jars equilibrated with a closed lid for 4 hours at standard room temperature and pressure. Malodor and candidate malodor reducing compound dosages had been previously determined by panelist dose-response experiments and the dosage corresponding to average mid-intensity was selected for both components. The malodor (MO) component abbreviations are 4MOA (4-methyl octanoic acid), 3H3MHA (3-methyl-3-hydroxyhexanoic acid), 3M2HA (3-methyl-2-hexenoic acid), 3M3SH (3-methyl-3-sulphanylhexanol).

TABLE 2

| | | | Test | | | |
|---|---|---|---|---|---|---|
| | | MO | Compound | | Significant | |
| | | Dosage | Dosage | Reduction | at | |
| Name Text | MO | (%) | (%) | (%) | a = 0.95 | N |
| ethyl | 4MO | 7.5 | 1.5 | 43 | * | 26 |
| tricyclo | A | | | | | |
| [5.2.1.0.(2,6)] | | | | | | |
| decane-2- | | | | | | |
| carboxylate | | | | | | |

### Example 3: Antiperspirant or Deodorant Compositions

Antiperspirant or deodorant compositions were prepared by admixing the following ingredients as indicated in Table 3 to Table 8:

TABLE 3
an antiperspirant or deodorant composition

| Ingredient | Amount |
|---|---|
| (3aRS,5aSR,9aSR,9bRS)-3a,6,6,9a- | 5.00% |
| tetramethyldodecahydronaphtho[2,1-b]furan | |
| 4-(4-hydroxyphenyl)-2-butanone | 3.00% |
| 2-(3-phenylpropyl)pyridine | 0.20% |
| 2-chromenone | 10.00% |
| 1-(3,3/5,5-dimethyl-1-cyclohexen-1-yl)-4-penten-1-one | 5.00% |
| 2-ethyl-3-hydroxy-4(4H)-pyranone | 1.00% |
| 3-ethoxy-4-hydroxybenzaldehyde | 2.00% |
| (+−)-tetrahydro-2-isobutyl-4-methyl-4(2H)-pyranol | 9.40% |
| (+−)-5-heptyldihydro-2(3H)-furanone | 5.00% |
| (E)-3,7-dimethyl-2,6-octadien-1-ol | 10.00% |
| (+−)-(3E)-3-methyl-4-(2,6,6-trimethyl- | 10.00% |
| 2-cyclohexen-1-yl)-3-buten-2-one, (+−)- | |
| (1E)-1-(2,6,6-trimethyl-2-cyclohexen-1- | |
| yl)-1-penten-3-one | |
| (+−)-3,5,6,6-tetramethyl-4-methylidene- | 5.00% |
| 2-heptanone, (4E)-3,4,5,6,6-pentamethyl-4- | |
| hepten-2-one, 3,4,5,6,6-pentamethyl- | |
| 3-hepten-2-one | |
| (+−)-(2,5-dimethyl-2,3-dihydro- | 10.00% |
| 1H-inden-2-yl)methanol | |
| 1-(2-naphthyl)ethanone | 5% |
| (+−)-3/4-(4-methyl-3-penten-1-yl)- | 5.00% |
| 3-cyclohexene-1-carbaldehyde | |
| methyl 2,4-dihydroxy-3,6-dimethylbenzoate | 2.00% |
| (+)-(3R,5Z)-3-methyl-5-cyclopentadecen-1-one | 0.10% |
| tricyclo[5.2.1.0(2,6)]dec-3-en-8-yl | 10.00% |
| propanoate (a) + tricyclo[5.2.1.0(2,6)]dec- | |
| 4-en-8-yl propanoate (b) | |
| (4Z)-4-dodecenal | 0.30% |
| thymol | 2.00% |

TABLE 4
an antiperspirant or deodorant composition

| Ingredient | Amount |
|---|---|
| 4-methoxybenzaldehyde | 18.00% |
| 10-undecenal | 2.50% |
| (3aRS,5aSR,9aSR,9bRS)-3a,6,6,9a- | 2.00% |
| tetramethyldodecahydronaphtho[2,1-b]furan | |
| Clearwood ® | 8.00% |
| 2-chromenone | 15.00% |
| (+−)-(3E)-3-methyl-4-(2,6,6-trimethyl- | 47.00% |
| 2-cyclohexen-1-yl)-3-buten-2-one, (+−)- | |
| (1E)-1-(2,6,6-trimethyl-2-cyclohexen-1- | |
| yl)-1-penten-3-one | |
| (E)-2-ethoxy-5-(1-propenyl)phenol | 1.50% |
| Ylang | 6.00% |

TABLE 5
an antiperspirant or deodorant composition

| Ingredient | Amount |
|---|---|
| 4-methoxybenzaldehyde | 18.00% |
| decanal | 12.50% |
| octanal | 3.00% |
| (+−)-2-methylundecanal | 1.50% |
| 4-(4-hydroxyphenyl)-2-butanone | 3.50% |
| (+−)-(2E)-1-(2,6,6-trimethyl-2-cyclohexen-1-yl)-2-buten-1-one | 0.50% |
| methyl 2,6,6-trimethyl-3-cyclohexene-1-carboxylate | 4.00% |
| litsea cubeba berry oil | 1.00% |
| tricyclo[5.2.1.0(2,6)]dec-3/4-en-8-yl propanoate | 18.00% |
| (+−)-(3E)-4-(2,6,6-trimethyl-1/2-cyclohexen-1-yl)-3-buten-2-one | 13.00% |
| (3E)-4-(2,6,6-trimethyl-1-cyclohexen-1-yl)-3-buten-2-one | 25.00% |

TABLE 6
an antiperspirant or deodorant composition

| Ingredient | Amount |
|---|---|
| 4-methoxybenzaldehyde | 2.50% |
| undecanal | 0.05% |
| (3aRS,5aSR,9aSR,9bRS)-3a,6,6,9a- | 1.00% |
| tetramethyldodecahydronaphtho[2,1-b]furan | |
| (3Z)-3-hexen-1-ol | 5.00% |
| (−)-(2E)-2-ethyl-4-[(1R)-2,2,3-trimethyl- | 3.00% |
| 3-cyclopenten-1-yl]-2-buten-1-ol | |
| (+−)-2,6-dimethyl-7-octen-2-ol | 10.00% |
| dipropylene glycol | 35.45% |
| (+−)-(2,5-dimethyl-2,3-dihydro-1H- | 4.00% |
| inden-2-yl)methanol | |
| 2-phenylethanol | 3.00% |
| (+−)-3-methyl-5-phenyl-1-pentanol | 5.00% |
| (E)-2-ethoxy-5-(1-propenyl)phenol | 7.00% |
| 1-[(1RS,2RS)-1,2,8,8-tetramethyl-1,2,3,4,5,6,7,8- | 20.00% |
| octahydro-2-naphthalenyl]ethenone, 1-((2RS,3RS)- | |
| 2,3,8,8-tetramethyl-1,2,3,4,5,6,7,8- | |
| octahydronaphthalen-2-yl)ethanone, 1-[(2RS,3RS,8aRS)- | |
| 2,3,8,8-tetramethyl-1,2,3,5,6,7,8,8a-octahydro-2- | |
| naphthalenyl]ethenone, 1-[(1RS,2RS,8aSR)-1,2,8,8- | |
| tetramethyl-1,2,3,5,6,7,8,8a-octahydro-2- | |
| naphthalenyl]ethenone, 1-[(2RS,3RS,8aRS)-2,3,8,8- | |
| tetramethyl-1,2,3,4,6,7,8,8a-octahydro-2-naphthalenyl]ethanone | |
| (3E)-4-(2,6,6-trimethyl-1-cyclohexen-1-yl)-3-buten-2-one | 4.00% |

TABLE 7
an antiperspirant or deodorant composition

| Ingredient | Amount |
|---|---|
| 4-methoxybenzaldehyde | 2.50% |
| undecanal | 0.05% |
| (+−)-2-methylundecanal | 2.00% |
| (3aRS,5aSR,9aSR,9bRS)-3a,6,6,9a- | 1.00% |
| tetramethyldodecahydronaphtho[2,1-b]furan | |
| 4-(4-hydroxyphenyl)-2-butanone | 10.00% |
| (3Z)-3-hexen-1-ol | 5.00% |
| (−)-(2E)-2-ethyl-4-[(1R)-2,2,3- | 3.00% |
| trimethyl-3-cyclopenten-1-y1]-2-buten-1-ol | |
| (+−)-2,6-dimethyl-7-octen-2-ol | 10.00% |
| dipropylene glycol | 18.45% |
| (+−)-(2,5-dimethyl-2,3-dihydro- | 4.00% |
| 1H-inden-2-yl)methanol | |
| 2-phenylethanol | 3.00% |
| (+−)-3-methyl-5-phenyl-1-pentanol | 5.00% |
| (E)-2-ethoxy-5-(1-propenyl)phenol | 7.00% |
| 1-[(1RS,2RS)-1,2,8,8-tetramethyl-1,2,3,4,5,6,7,8- | 20.00% |
| octahydro-2-naphthalenyl]ethenone, 1-((2RS,3RS)- | |
| 2,3,8,8-tetramethyl-1,2,3,4,5,6,7,8- | |
| octahydronaphthalen-2-yl)ethanone, 1-[(2RS,3RS,8aRS)- | |
| 2,3,8,8-tetramethyl-1,2,3,5,6,7,8,8a-octahydro-2- | |
| naphthalenyl]ethenone, 1-[(1RS,2RS,8aSR)-1,2,8,8- | |
| tetramethyl-1,2,3,5,6,7,8,8a-octahydro-2- | |
| naphthalenyl]ethenone, 1-[(2RS,3RS,8aRS)-2,3,8,8- | |
| tetramethyl-1,2,3,4,6,7,8,8a-octahydro-2- | |
| naphthalenyl]ethanone | |
| (1RS,2RS)-2,4-dimethyl-3-cyclohexene- | 5.00% |
| 1-carbaldehyde, (1RS,2SR)-2,4-dimethyl- | |
| 3-cyclohexene-1-carbaldehyde | |
| (3E)-4-(2,6,6-trimethyl-1-cyclohexen-1- | 4.00% |
| yl)-3-buten-2-one | |

TABLE 8
an antiperspirant or deodorant composition

| Ingredient | Amount |
|---|---|
| (3aRS,5aSR,9aSR,9bRS)-3a,6,6,9a- | 5.00% |
| tetramethyldodecahydronaphtho[2,1-b]furan | |
| Clearwood ® | 15.00% |
| 2-chromenone | 10.00% |
| dipropylene glycol | 2.97% |
| 1-(3,3/5,5-dimethyl-1-cyclohexen-1-yl)-4-penten-1-one | 5.00% |
| 3-ethoxy-4-hydroxybenzaldehyde | 2.00% |
| 2-methoxy-4-(2-propen-1-yl)phenol | 5.00% |
| (+−)-5-heptyldihydro-2(3H)-furanone | 5.00% |
| methyl 2-((1RS,2RS)-3-oxo-2-pentylcyclopentyl)acetate | 10.00% |
| 6/8-sec-butylquinoline | 1.00% |
| (+−)-(3E)-3-methyl-4-(2,6,6-trimethyl- | 12.40% |
| 2-cyclohexen-1-yl)-3-buten-2-one, (+−)- | |
| (1E)-1-(2,6,6-trimethyl-2-cyclohexen-1- | |
| yl)-1-penten-3-one | |
| methyl 2,4-dihydroxy-3,6-dimethylbenzoate | 4.00% |
| (+)-(3R,5Z)-3-methyl-5-cyclopentadecen-1-one | 0.10% |
| (2E,6Z)-2,6-nonadienal | 0.03% |
| tricyclo[5.2.1.0(2,6)]dec- | 10.00% |
| 3/4-en-8-yl propanoate | |
| (4Z)-4-dodecenal | 0.50% |
| thymol | 2.00% |
| (+−)-(E)-4-methyl-3-decen-5-ol | 5.00% |
| (+−)-(3E)-4-(2,6,6-trimethyl-1/2- | 5.00% |
| cyclohexen-1-yl)-3-buten-2-one | |

### Example 4: Preparation of Anhydrous Antiperspirant Spray Formulations Comprising a Invention's Composition

A typical unperfumed anhydrous antiperspirant spray formulation is listed in Table 9. The anhydrous antiperspirant spray formulation is prepared by using a high speed stirrer. Silica and Quaternium-18-hectorite are added to the mixture of isopropyl myristate and cyclomethicone. Once completely swollen, aluminium chlorohydrate is added portion-wise under stirring until the mixture becomes homogeneous and without lumps.

TABLE 9
Composition of a typical unperfumed
anhydrous antiperspirant spray.

| Ingredients | Amount [wt %] |
|---|---|
| Cyclomethicone ⁽¹⁾ | 53.51 |
| Isopropyl myristate | 9.04 |
| Silica ⁽²⁾ | 1.03 |
| Quaternium-18-hectorite ⁽³⁾ | 3.36 |
| Aluminium chlorohydrate ⁽⁴⁾ | 33.06 |

⁽¹⁾ Dow Corning ® 345 Fluid; origin: Dow Corning
⁽²⁾ Aerosil ® 200 ; origin: Evonik
⁽³⁾ Bentone ® 38; origin: Elementis Specialities
⁽⁴⁾ Micro Dry Ultrafine; origin: Reheis

The perfumed formulation is then obtained by adding one of the antiperspirant or deodorant compositions of Example 3 (0.1 to 1% by weight relative to the total weight of the antiperspirant spray formulation) into the unperfumed antiperspirant spray formulation of Table 9.

The aerosol cans are filled with 25% Suspension of the suspension and 75% of Propane/Butane (2,5 bar).

### Example 5: Preparation of Deodorant Spray Emulsion Formulations Comprising an Invention's Composition

A typical deodorant spray emulsion formulation is prepared by mixing and dissolving all the ingredients according to the sequence of Table 10. Then one of the antiperspirant or deodorant compositions of Example 3 (0.1 to 1.5% by weight relative to the total weight of the deodorant spray formulation) is added under gentle shaking. Then aerosol cans are filled, and the propellant is crimped and added. Aerosol filling: 40% active solution 60% propane/butane (2.5 bar).

TABLE 10
Composition of a typical unperfumed deodorant spray formulation.

| Ingredients | Amount [wt %] |
|---|---|
| Ethanol (95%) | 90.65 |
| Triclosan ⁽¹⁾ | 0.26 |
| Isopropyl myristate | 9.09 |

⁽¹⁾ Irgasan ® DP 300; origin: BASF

### Example 6: Preparation of Deodorant Stick Formulations Comprising an Invention's Composition

A typical unperfumed deodorant stick formulation is listed in Table 11. The deodorant stick formulation is obtained by weighing all the components of Part A and heating to 70-75° C. Ceteareth-25 is added once the other Part A ingredients are mixed and heated. Once the Ceteareth-25 is dissolved, stearic acid is added. Part B is prepared by dissolving Triclosan in 1,2-propylene glycol. Evaporated water is compensated. Then, slowly, under mixing, Part B is poured into Part A.

TABLE 11
Composition of a typical unperfumed deodorant stick formulation.

| Phase | Ingredients | Amount [wt %] |
|---|---|---|
| A | Stearic acid | 5.05 |
| | 1,2-Propylene glycol | 41.87 |
| | Sodium hydroxide (20% aqueous solution) | 4.24 |
| | Water | 30.30 |
| | Tetrasodium EDTA ⁽¹⁾ | 0.10 |
| | Ceteareth-25 ⁽²⁾ | 1.52 |
| | PPG-3 Myristyl ether ⁽³⁾ | 1.52 |
| B | 1,2-Propylene glycol | 15.14 |
| | Triclosan ⁽⁴⁾ | 0.25 |

⁽¹⁾ Edeta ® B Power; origin: BASF
⁽²⁾ Cremophor ® A25; origin: BASF
⁽³⁾ Tegosoft ® APM; origin: Evonik
⁽⁴⁾ Irgasan ® DP 300; origin: BASF

The perfumed deodorant stick formulation is then obtained by adding one of the antiperspirant or deodorant compositions of Example 3 (0.1 to 1.5% by weight relative to the total weight of the deodorant stick formulation) under gentle shaking. To stock, a plastic bag is put into the bucket to be sealed after cooling. Moulds were filled at about 70° C.

### Example 7: Preparation of Deodorant Roll-on Formulations Comprising an Invention's Composition

A typical unperfumed deodorant roll-on formulation is listed in Table 12. Part A is prepared by sprinkling little-by-little the hydroxyethylcellulose into the water, whilst rapidly stirring with a turbine until the hydroxyethylcellulose is entirely swollen giving a limpid gel. Part B is slowly poured into Part A, whilst continuing stirring until the entire mixture is homogeneous. Then Part C is added.

TABLE 12
Composition of a typical unperfumed
deodorant roll-on formulation.

| Phase | Ingredients | Amount [wt %] |
|---|---|---|
| A | Water | 50.51 |
| | Hydroxyethylcellulose ⁽¹⁾ | 0.71 |
| B | Ethanol (95%) | 40.40 |
| | 1,2-Propylene glycol | 5.05 |
| | Triclosan ⁽²⁾ | 0.30 |
| C | PEG-40 hydrogenated castor oil ⁽³⁾ | 3.03 |

⁽¹⁾ Natrosol ® 250 H; origin: Ashland
⁽²⁾ Irgasan ® DP 300; origin: BASF
⁽³⁾ Cremophor ® RH 40; origin: BASF

The perfumed deodorant roll-on formulation is then obtained by adding one of the antiperspirant or deodorant compositions of Example 3 (0.1 to 1% by weight relative to the total weight of the deodorant stick formulation) under gentle shaking.

### Example 8: Sensory Evaluation of a Spray Deodorant Formulation Comprising the Invention's Antiperspirant or Deodorant Composition

A test formulation as reported in Example 5 comprising invention's composition 1 corresponding to composition reported in Table 5 was generated. 0.8 gram (+/−0.05 gram) of the test formulation was applied to separate large blotters (5 cm width) from a distance of 12 cm from the aerosol can. The aerosol can was shaken before each spray. In parallel, a control composition 2, a composition that was not comprised of compounds cited in this application, was similarly applied to separate large blotters. Each blotter was placed on a 35° C. hot plate for 6 hours.

A minimum of 20 skilled panelists were asked to evaluate the intensity of the fragrance of the formulation and the intensity of the perceived malodour, in a blind, balanced, randomized study, with a 60 second break between samples.

35 minutes prior to the study, 1.2 grams of sweat body odour solution was applied to a filter paper placed at the bottom of a 500 mL glass jar. 20 minutes prior to the study, each blotter with a fragrance formulation was placed into each related jar. Jars were placed into a 40° C. oven for 15 minutes. 5 minutes prior to the study, jars were taken out of the oven and left to cool for 5 minutes. The panelists were asked to (i) rank the intensity of the fragrance from 0 (not perceptible), to 10 (very strong odour), and (ii) rank the intensity of the perceived malodour from 0 (not perceptible), to 10 (very strong odour). The results of the evaluation were analyzed using Analysis of Variance with Duncan's post-hoc analysis (α=0.05), wherein:

$$\% MO reduction = \frac{( {MO}_{negative control} - {MO}_{test sample} ) \times 100}{{MO}_{negative control} - {MO}_{positive control}}$$

The results are shown in Table 13 below.

TABLE 13
Percentage reduction in the perceived malodour intensity 6
hours after application of a spray deodorant formulation comprising
invention's composition 1 or control composition 2

| | Malodour intensity perceived at 6 hours |
|---|---|
| Composition | after application |
| invention's composition 1 | 71% |
| control composition 2 | 18% |

Referring to Table 13, the test formulation comprising invention's composition 1 demonstrated a greater percentage reduction in the perceived malodour intensity 6 hours after application than the control composition 2.

1. A method to inhibit, reduce, or suppress the perception of a sweat malodor in a subject, which comprises contacting the subject with a composition comprising a compound selected from the group consisting of:
(+−)-2,6-dimethyl-2-octanol; allyl heptanoate; (2E)-3-phenyl-2-prop en-1-ol; (+−)-2,2,5-trimethyl-5-pentylcyclopentanone; (+−)-3,7-dim ethyl-3-octanol; methyl 2-octynoate; (+−)-2-methylundecanal; (+−)-methyl 2,2-dimethyl-6-methylidenecyclohexanecarboxylate; ethyl (E)-3-phenyl-2-propenoate; methyl (E)-3-phenyl-2-propenoate; 1-(2-naphthyl)ethenone; (10E)-oxacycloheptadec-10-en-2-one; 4-(4-hydroxyphenyl)-2-butanone; (+−)-6-m ethoxy-2,6-dimethylheptanal; ethyltricyclo[5.2.1.0.(2,6)] decane-2-carboxylate; 4-methoxybenzyl acetate; (3Z)-3-hexen-1-ol; pentyl 2-hydroxybenzoate; allyl hexanoate; tricyclo[5.2.1.0(2,6)] dec-3-en-8-yl propanoate; (2RS,4SR)-4-methyl-2-phenyltetrahydro-2H-pyran; (+−)-3-(4-m ethoxyphenyl)-2-m ethylprop anal; (+−)-3-(1,3-benzodioxol-5-yl)-2-m ethylprop anal; 2,6-dim ethyl-4-heptanol; benzophenone; (2Z)-1,1-dimethoxy-3,7-dimethyl-2,6-octadiene; 4-isopropylbenzaldehyde; (+−)-4-nonanolide; (E)-4-decenal; (Z)-3,7-dimethyl-1,3,6-octatriene; 3-phenylpropanal; 2-methoxynaphthalene; 2-ethyl-3-hydroxy-4(4H)-pyranone; (+−)-2-(4-methyl-3-cyclohexen-1-yl)-2-propanyl acetate; (E)-3,7-dim ethyl-2,6-octadien-1-ol; ethyl 2-methyl-1,3-di oxolane-2-acetate; 3-(4-ethylphenyl)-2,2-dimethylpropanal; (2RS,4 SR)-4-methyl-2-(2-methyl-1-prop en-1-yl)tetrahydro-2H-pyran; (3 aRS, 5 aSR,9aSR,9bRS)-3a,6,6,9a-tetramethyl dodecahydronaphtho[2,1-b] furan; (2,2-dimethoxyethyl)benzene; (+−)-(5E)-3-methyl-4-methoxybenzaldehyde; 10-undecenal; (2E)-2-nonenal; 4-phenyl-2-butanone; (+−)-(6E)-3,7-dim ethyl-1,6-nonadien-3-yl acetate; ethyl 1RS,2 SR, 3 SR,4 SR-3-isopropylbicyclo[2.2.1] hept-5-ene-2-carboxylate; 3-ethoxy-4-hydroxybenzaldehyde; methyl 2-((1RS,2RS)-3-oxo-2-pentylcyclopentyl)acetate; (+−)-2,6-dimethyl-7-octen-2-ol; (+−)-6-sec-butyl quinoline; (+−)-3,7-dimethyl-6-octenal; methyl 2,4-dihydroxy-3,6-dimethylbenzoate; 4-cyclohexyl-2-methyl-2-butanol; (3E)-4-(2,6,6-trimethyl-1-cyclohexen-1-yl)-3-buten-2-one; (−)-(2E)-2-ethyl-4-[(1R)-2,2,3-tri methyl-3-cyclopenten-1-yl]-2-buten-1-ol; 1-(3, 3/5, 5-di methyl-1-cyclohexen-1-yl)-4-penten-1-one; (+)-(3R,5Z)-3-methyl-5-cyclopentadecen-1-one; patchouli oil; thymol; (+−)-(3E)-4-(2,6,6-trimethyl-1/2-cyclohexen-1-yl)-3-buten-2-one; 2-(3-phenyl propyl)pyri dine; (+−)-(2E)-1-(2,6,6-tri methyl-2-cyclohexen-1-yl)-2-buten-1-one; (+−)-3, 5,6,6-tetramethyl-4-methylidene-2-heptanone; (4E)-3,4, 5,6,6-pentamethyl-4-hepten-2-one; 3,4, 5,6,6-pentamethyl-3-hepten-2-one; (2E,6Z)-2,6-nonadienal; (+−)-(E)-4-methyl-3-dec en-5-ol; 2-chromenone; (+−)-(3E)-3-methyl-4-(2,6,6-trimethyl-2-cyclohexen-1-yl)-3-buten-2-one; (+−)-(1E)-1-(2,6,6-tri methyl-2-cyclohexen-1-yl)-1-penten-3-one; (+−)-(2,5-di methyl-2,3-dihydro-1H-inden-2-yl)methanol; (E)-2-ethoxy-5-(1-propenyl)phenol; undecanal; 1-[(1RS,2RS)-1,2, 8,8-tetramethyl-1,2,3,4, 5, 6,7,8-octahydro-2-naphthalenyl] ethenone; 1-((2RS,3RS)-2,3, 8,8-tetramethyl-1,2,3,4, 5, 6,7,8-octahydronaphthalen-2-yl)ethenone; 1-[(2RS, 3 RS, 8 aRS)-2,3, 8,8-tetramethyl-1,2,3, 5,6,7,8,8a-octahydro-2-naphthalenyl] ethenone; 1-[(1RS,2RS, 8aSR)-1,2, 8,8-tetramethyl-1,2,3, 5,6,7,8,8a-octahydro-2-naphthalenyl] ethenone; 1-[(2RS, 3 RS, 8 aRS)-2,3, 8,8-tetramethyl-1,2,3,4,6, 7, 8,8a-octahydro-2-naphthalenyl] ethenone; 2-phenylethanol, (4Z)-4-dodecenal; octanal; decanal; (+−)-tetrahydro-2-isobutyl-4-methyl-4(2H)-pyranol; (1RS,2RS)-2,4-dimethyl-3-cyclohexene-1-carb aldehyde, (1RS,2SR)-2,4-dimethyl-3-cyclohexene-1-carbaldehyde; and combinations thereof;
wherein the subject's perception of the sweat malodor is inhibited, reduced or suppressed.

2. The method of claim 1, wherein the composition comprises (3E)-4-(2,6,6-trimethyl-1-cyclohexen-1-yl)-3-buten-2-one in an amount of at least at least 6 wt. %, based on the total weight of the composition; and/or
(−)-(2E)-2-ethyl-4-[(1R)-2,2,3-trimethyl-3-cyclopenten-1-yl]-2-buten-1-ol in an amount of at least at least 7 wt. %, based on the total weight of the composition; and/or
1-(3,3/5,5-dimethyl-1-cyclohexen-1-yl)-4-penten-1-one in an amount of at least at least 0.4 wt. %, based on the total weight of the composition; and/or
(+)-(3R,5Z)-3-methyl-5-cyclopentadecen-1-one in an amount of at least at least 0.6 wt. %, based on the total weight of the composition; and/or
patchouli oil in an amount of at least at least 3 wt. %, based on the total weight of the composition; and/or
thymol in an amount of at least at least 0.5 wt. %, based on the total weight of the composition; and/or
(+−)-(3E)-4-(2,6,6-trimethyl-1/2-cyclohexen-1-yl)-3-buten-2-one in an amount of at least at least 4 wt. %, based on the total weight of the composition; and/or
2-(3-phenylpropyl)pyridine in an amount of at least at least 0.0007 wt. %, based on the total weight of the composition; and/or
(+−)-(2E)-1-(2,6,6-trimethyl-2-cyclohexen-1-yl)-2-buten-1-one in an amount of at least at least wt. %, based on the total weight of the composition; and/or
(+−)-3,5,6,6-tetramethyl-4-methylidene-2-heptanone, (4E)-3,4,5,6,6-pentamethyl-4-hepten-2-one and 3,4,5,6,6-pentamethyl-3-hepten-2-one in an amount of at least at least 6 wt. %, based on the total weight of the composition; and/or
(2E,6Z)-2,6-nonadienal in an amount of at least at least 0.004 wt. %, based on the total weight of the composition; and/or
(+−)-(E)-4-methyl-3-decen-5-ol in an amount of at least at least 5 wt. %, based on the total weight of the composition; and/or
2-chromenone in an amount of at least at least 9 wt. %, based on the total weight of the composition; and/or
(+−)-(3E)-3-methyl-4-(2,6,6-trimethyl-2-cyclohexen-1-yl)-3-buten-2-one and (+−)-(1E)-1-(2,6,6-trimethyl-2-cyclohexen-1-yl)-1-penten-3-one in an amount of at least at least 7 wt. %, based on the total weight of the composition; and/or
(+−)-(2,5-dimethyl-2,3-dihydro-1H-inden-2-yl)methanol in an amount of at least at least 4 wt. %, based on the total weight of the composition; and/or
(E)-2-ethoxy-5-(1-propenyl)phenol; in an amount of at least at least 0.005 wt. %, based on the total weight of the composition; and/or
undecanal in an amount of at least at least 3 wt. %, based on the total weight of the composition; and/or
1-[(1RS,2RS)-1,2,8,8-tetramethyl-1,2,3,4,5,6,7,8-octahydro-2-naphthalenyl]ethenone, 1-((2RS,3RS)-2,3,8,8-tetramethyl-1,2,3,4,5,6,7,8-octahydronaphthalen-2-yl)ethenone, 1-[(2RS,3RS,8aRS)-2,3,8,8-tetramethyl-1,2,3,5,6,7,8,8a-octahydro-2-naphthalenyl]ethenone, 1-[(1RS,2RS,8aSR)-1,2,8,8-tetramethyl-1,2,3,5,6,7,8,8a-octahydro-2-naphthalenyl]ethenone, 1-[(2RS,3RS,8aRS)-2,3,8,8-tetramethyl-1,2,3,4,6,7,8,8a-octahydro-2-naphthalenyl]ethenone in an amount of at least at least 8 wt. %, based on the total weight of the composition; and/or
2-phenylethanol in an amount of at least at least 20 wt. %, based on the total weight of the composition; and/or
(4Z)-4-dodecenal in an amount of at least at least 0.01 wt. %, based on the total weight of the composition; and/or
octanal in an amount of at least at least 4 wt. %, based on the total weight of the composition; and/or
decanal in an amount of at least at least 5 wt. %, based on the total weight of the composition; and/or
(+−)-tetrahydro-2-isobutyl-4-methyl-4(2H)-pyranol in an amount of at least at least 20 wt. %, based on the total weight of the composition; and/or;
(1RS,2RS)-2,4-dimethyl-3-cyclohexene-1-carbaldehyde and (1RS,2SR)-2,4-dimethyl-3-cyclohexene-1-carbaldehyde in an amount of at least at least 2 wt. %, based on the total weight of the composition;
wherein the subject's perception of the sweat malodor is inhibited, reduced, or suppressed.

3. The method of claim 1, wherein the composition comprises from about 1% to about 100% of (+−)-2,2,5-trimethyl-5-pentylcyclopentanone;
wherein the subject's perception of the sweat malodor is inhibited, reduced, or suppressed.

4. The method of claim 1, wherein the composition comprises from about 2% to about 100% of a compound selected from the group consisting of: (2E)-3-phenyl-2-propen-1-ol; methyl (E)-3-phenyl-2-propenoate; 1-(2-naphthyl)ethenone; (10E)-oxacycloheptadec-10-en-2-one; and combinations thereof;
wherein the subject's perception of the sweat malodor is inhibited.

5. The method of claim 1, wherein the composition comprises from about 3% to about 100% of a compound selected from the group consisting of: (+−)-2,6-dimethyl-2-octanol; 4-methoxybenzyl acetate; tricyclo[5.2.1.0(2,6)]dec-3/4-en-8-yl propanoate; and combinations thereof;
wherein the subject's perception of the sweat malodor is inhibited, reduced or suppressed.

6. The method of claim 1, wherein the composition comprises from about 0.1% to about 100% of a compound selected from the group consisting of: (2RS,4SR)-4-methyl-2-phenyltetrahydro-2H-pyran; 3-(6,6-dimethyl-bicyclo[3.1.1]hept-2-en-2-yl)propanal; and
combinations thereof;
wherein the subject's perception of the sweat malodor is inhibited, reduced or suppressed.

7. The method of claim 1, wherein the composition comprises from about 7% to about 100% of a compound selected from the group consisting of: 4-(4-hydroxyphenyl)-2-butanone; allyl heptanoate; and combinations thereof;
wherein the subject's perception of the sweat malodor is inhibited, reduced or suppressed.

8. The method of claim 1, wherein the composition comprises from about 20% to about 100% of a compound selected from the group consisting of: pentyl 2-hydroxybenzoate;
(+−)-3,7-dimethyl-3-octanol; and combinations thereof;
wherein the subject's perception of the sweat malodor is inhibited, reduced or suppressed.

9. The method of claim 1, wherein the composition comprises from about 4% to about 100% of (+−)-3-(1,3-benzodioxol-5-yl)-2-methyl prop anal; wherein the subject's perception of the sweat malodor is inhibited, reduced or suppressed.

10. The method of claim 1, wherein the composition comprises from about 0.06% to about 100% of methyl 2-octynoate; wherein the subject's perception of the sweat malodor is inhibited, reduced or suppressed.

11. The method of claim 1 wherein the composition comprises from about 0.4% to about 100% of (+−)-methyl 2,2-dimethyl-6-methylidenecyclohexanecarboxylate; wherein the subject's perception of the sweat malodor is inhibited, reduced or suppressed.

12. The method of claim 1, wherein the composition comprises from about 0.7% to about 100% of ethyl (E)-3-phenyl-2-propenoate; wherein the subject's perception of the sweat malodor is inhibited, reduced or suppressed

13. The method of claim 1, wherein the composition comprises about 0.6% to about 100% of ethyl tricyclo[5.2.1.0.(2,6)]decane-2-carboxylate; wherein the subject's perception of the sweat malodor is inhibited, reduced or suppressed.

14. The method of claim 1, wherein the composition further comprises a perfume ingredient.

15. A sweat malodor inhibiting composition comprising a compound selected from the group consisting of:
(+−)-2,6-dim ethyl-2-octanol allyl heptanoate; (2E)-3-phenyl-2-prop en-1-ol (+−)-2,2,5-trimethyl-5-pentylcyclopentanone; (+−)-3,7-dim ethyl-3-octanol; methyl 2-octynoate; (+−)-2-methylundecanal; (+−)-methyl 2,2-dimethyl-6-methylidenecyclohexanecarboxylate; ethyl (E)-3-phenyl-2-propenoate; methyl (E)-3-phenyl-2-propenoate; 1-(2-naphthyl)ethenone; (10E)-oxacycloheptadec-10-en-2-one; 4-(4-hydroxyphenyl)-2-butanone; (+−)-6-methoxy-2,6-dimethylheptanal; ethyltricyclo [5.2.1.0.(2,6)]decane-2-carboxylate; 4-methoxybenzyl acetate; (3Z)-3-hexen-1-ol; pentyl 2-hydroxybenzoate; allyl hexanoate; tricyclo[5.2.1.0(2,6)]dec-3-en-8-yl propanoate; (2RS,4SR)-4-methyl-2-phenyltetrahydro-2H-pyran; (+−)-3-(4-m ethoxyphenyl)-2-methyl prop anal; (+−)-3-(1,3-benzodioxol-5-yl)-2-methyl prop anal; 2,6-dim ethyl-4-heptanol benzophenone; (2Z)-1,1-dimethoxy-3,7-dimethyl-2,6-octadiene; 4-isopropylbenzaldehyde; (+−)-4-nonanolide; (E)-4-decenal; (Z)-3,7-dimethyl-1,3,6-octatriene; 3-phenylpropanal; 2-methoxynaphthalene; 2-ethyl-3-hydroxy-4(4H)-pyranone; (+−)-2-(4-methyl-3-cyclohexen-1-yl)-2-propanyl acetate; (E)-3,7-dim ethyl-2,6-octadien-1-ol; ethyl 2-methyl-1,3-dioxolane-2-acetate; 3-(4-ethylphenyl)-2,2-dimethylpropanal; (2RS,4 SR)-4-methyl-2-(2-methyl-1-prop en-1-yl)tetrahydro-2H-pyran; (3 aRS, 5 aSR,9aSR,9bRS)-3a,6,6,9a-tetramethyl dodecahydronaphtho[2,1-b]furan; (2,2-dimethoxyethyl)benzene; (+−)-(5E)-3-methyl-5-cyclopentadecen-1-one; 4-methoxybenzaldehyde; 10-undecenal; (2E)-2-nonenal; 4-phenyl-2-butanone; (+−)-(6E)-3,7-dimethyl-1,6-nonadien-3-yl acetate; ethyl 1RS,2SR,3 SR,4 SR-3-isopropylbicyclo[2.2.1]hept-5-ene-2-carboxylate; 3-ethoxy-4-hydroxybenzaldehyde; methyl 2-((1RS,2RS)-3-oxo-2-pentylcyclopentyl)acetate; (+−)-2,6-dimethyl-7-octen-2-ol; (+−)-6-sec-butyl quinoline; (+−)-3,7-dimethyl-6-octenal; methyl 2,4-dihydroxy-3,6-dimethylbenzoate; 4-cyclohexyl-2-methyl-2-butanol (3E)-4-(2,6,6-trimethyl-1-cyclohexen-1-yl)-3-buten-2-one; (−)-(2E)-2-ethyl-4-[(1R)-2,2,3-trimethyl-3-cyclopenten-1-yl]-2-buten-1-ol; 1-(3,3/5, 5-dimethyl-1-cyclohexen-1-yl)-4-penten-1-one; (+)-(3R,5Z)-3-methyl-5-cyclopentadecen-1-one; patchouli oil; thymol; (+−)-(3E)-4-(2,6,6-trimethyl-1/2-cyclohexen-1-yl)-3-buten-2-one; 2-(3-phenylpropyl)pyri dine; (+−)-(2E)-1-(2,6,6-trimethyl-2-cyclohexen-1-yl)-2-buten-1-one; (+−)-3, 5,6,6-tetramethyl-4-methylidene-2-heptanone; (4E)-3,4,5,6,6-pentamethyl-4-hepten-2-one; 3,4, 5,6,6-pentamethyl-3-hepten-2-one; (2E,6Z)-2, 6-nonadienal (+−)-(E)-4-methyl-3-dec en-5-ol 2-chromenone; (+−)-(3E)-3-methyl-4-(2, 6,6-trimethyl-2-cyclohexen-1-yl)-3-buten-2-one; (+−)-(1E)-1-(2,6, 6-trimethyl-2-cyclohexen-1-yl)-1-penten-3-one; (+−)-(2, 5-dim ethyl-2,3-dihydro-1H-inden-2-yl)methanol (E)-2-ethoxy-5-(1-propenyl)phenol; undecanal; 1-[(1RS,2RS)-1,2, 8, 8-tetramethyl-1,2,3,4,5, 6,7, 8-octahydro-2-naphthalenyl] ethenone; 1-((2RS,3RS)-2,3, 8, 8-tetramethyl-1,2,3,4,5, 6,7, 8-octahydronaphthalen-2-yl)ethenone; 1-[(2RS,3RS, 8 aRS)-2,3, 8, 8-tetramethyl-1,2,3,5,6,7, 8, 8a-octahydro-2-naphthalenyl] ethenone; 1-[(1RS,2RS, 8aSR)-1,2, 8, 8-tetramethyl-1,2,3,5,6,7, 8, 8a-octahydro-2-naphthalenyl] ethenone; 1-[(2RS,3RS, 8 aRS)-2,3, 8, 8-tetramethyl-1,2,3,4,6,7, 8, 8a-octahydro-2-naphthalenyl] ethenone; 2-phenylethanol, (4Z)-4-dodecenal; octanal; decanal; (+−)-tetrahydro-2-isobutyl-4-methyl-4(2H)-pyranol; (1RS,2RS)-2,4-dimethyl-3-cyclohexene-1-carb aldehyde, (1RS,2SR)-2,4-dimethyl-3-cyclohexene-1-carbaldehyde; and combinations thereof;
and a perfume ingredient or an excipient selected from the group consisting of: a humectant, an emollient an antioxidant, a pH adjustor, a coloring agent, a preservative, a thickening agent, a stabilizer, a propellant, a solvent, a cooling agent, and combinations thereof.

16. (canceled)

17. (canceled)

18. (canceled)

19. (canceled)

20. An antiperspirant or deodorant composition comprising:
a) at least one ingredient selected from the group consisting of (3E)-4-(2,6,6-trimethyl-1-cyclohexen-1-yl)-3-buten-2-one, (+−)-2-methylundecanal, (−)-(2E)-2-ethyl-4-[(1R)-2,2,3-trimethyl-3-cyclopenten-1-yl]-2-buten-1-ol, 1-(3,3/5,5-dim ethyl-1-cyclohexen-1-yl)-4-penten-1-one, methyl 2,4-dihydroxy-3,6-dimethylbenzoate, (+)-(3R,5Z)-3-methyl-5-cyclopentadecen-1-one, patchouli oil, thymol, (+−)-(3E)-4-(2,6, 6-trimethyl-1/2-cyclohexen-1-yl)-3-buten-2-one, 2-(3-phenylpropyl)pyri dine, (+−)-(2E)-1-(2, 6,6-trimethyl-2-cyclohexen-1-yl)-2-buten-1-one, (+−)-6/8-sec-butyl quinoline, (+−)-3,5, 6,6-tetramethyl-4-methylidene-2-heptanone, (4E)-3,4, 5,6, 6-pentamethyl-4-hepten-2-one, 3,4,5,6,6-pentamethyl-3-hepten-2-one, 1-(2-naphthyl)ethanone, methyl 2-((1RS,2RS)-3-oxo-2-pentylcyclopentyl)acetate, (2E,6Z)-2,6-nonadienal, (+−)-(E)-4-methyl-3-decen-5-ol and combinations thereof;
b) at least two ingredients and selected from the group consisting of (3 aRS, 5aSR,9aSR,9bRS)-3a,6,6,9a-tetramethyldodecahydronaphtho[2, 1-b]furan, 4-methoxybenzaldehyde, 4-(4-hydroxyphenyl)-2-butanone, tricyclo[5.2.1.0(2,6)]dec-3/4-en-8-yl propanoate, 2-chromenone, (+−)-(3E)-3-methyl-4-(2,6,6-trimethyl-2-cyclohexen-1-yl)-3-buten-2-one, (+−)-(1E)-1-(2,6,6-trimethyl-2-cyclohexen-1-yl)-1-penten-3-one, (+−)-(2, 5-dimethyl-2,3-dihydro-1H-inden-2-yl)methanol, (E)-2-ethoxy-5-(1-prop enyl)phenol, undecanal, 1-[(1RS,2RS)-1,2,8, 8-tetramethyl-1,2,3,4, 5,6,7, 8-octahydro-2-naphthalenyl] ethenone, 1-((2RS,3RS)-2,3, 8, 8-tetramethyl-1,2,3,4, 5,6,7, 8-octahydronaphthalen-2-yl)ethanone, 1-[(2RS, 3RS, 8 aRS)-2,3, 8, 8-tetramethyl-1,2,3,5,6,7,8,8a-octahydro-2-naphthalenyl]ethenone, 1-[(1RS,2RS,8aSR)-1,2, 8,8-tetramethyl-1,2,3,5,6,7,8,8a-octahydro-2-naphthalenyl]ethenone, 1-[(2RS,3RS,8aRS)-2,3,8, 8-tetramethyl-1,2,3,4, 6,7, 8,8a-octahydro-2-naphthalenyl] ethanone, (+−)-2,6-dimethyl-7-octen-2-ol, 2-phenylethanol, (3Z)-3-hexen-1-ol, (4Z)-4-dodecenal, octanal, decanal, 10-undecenal, 3-ethoxy-4-hydroxybenzaldehyde, 2-ethyl-3-hydroxy-4(4H)-pyranone, (E)-3,7-dim ethyl-2,6-octadien-1-ol, (+−)-tetrahydro-2-isobutyl-4-methyl-4(2H)-pyranol, (1RS,2RS)-2,4-dimethyl-3-cyclohexene-1-carb aldehyde, (1RS,2SR)-2,4-dimethyl-3-cyclohexene-1-carbaldehyde, (+−)-3/4-(4-methyl-3-penten-1-yl)-3-cyclohexene-1-carbaldehyde and combinations thereof.
